(19) United States
(12) Patent Application Publication
(10) Pub. No.: US 2021/0196676 A1
(43) Pub. Date: Jul. 1, 2021

(54) TREATMENT OF VIRAL INFECTIONS AND VIRALLY ASSOCIATED LESIONS WITH SEQUITERPENE LACTONES

(71) Applicant: The Board of Regents of the University of Texas System, Austin, TX (US)

(72) Inventor: Ellen S. VITETTA, Dallas, TX (US)

(73) Assignee: The Board of Regents of the University of Texas System, Austin, TX (US)

(21) Appl. No.: 17/058,380

(22) PCT Filed: May 30, 2019
(86) PCT No.: PCT/US2019/034520

§ 371 Date: Nov. 24, 2020

Related U.S. Application Data
(60) Provisional application No. 62/677,928, filed on May 30, 2018.

(51) Int. Cl. A61K 31/365; A61K 9/00; A61K 9/06; A61K 31/60; A61K 31/19; A61K 31/513; A61K 45/06; A61K 31/122; A61K 31/4745; A61K 31/4164; A61K 9/70; A61P 31/22; A61F 13/40; A61M 35/00; A61B 18/02; A61B 18/20; A61B 18/14; A61B 17/3205

(52) U.S. Cl. CPC A61K 31/365; A61B 2018/00577; A61K 9/06; A61K 31/60; A61K 31/19; A61K 31/513; A61K 45/06; A61K 31/122; A61K 31/4745; A61K 31/4164; A61K 9/7023; A61P 31/22; A61M 35/006; A61M 35/10; A61B 18/02; A61B 18/20; A61B 18/14; A61B 17/3205; A61K 9/0014

(57) ABSTRACT

The present disclosure relates to the use of the sequiterpene lactones and Helenalin (H. tox) or its derivatives to treat viral infection and virally induced lesions, such as warts or wart-associated skin or mucosal lesion. A topical application, optionally using a skin covering such as gauze, a bandage, or a patch, can substantially reduce and even eliminate persistent, recurrent and/or treatment-resistant warts.

### PRIORITY INFORMATION

This application claims benefit of priority to U.S. Provisional Application No. 62/677,928, filed May 30, 2018, the entire contents of which are hereby incorporated by reference.

### BACKGROUND

### A. Field

The present disclosure relates to the fields of biology, virology, medicine and dermatology. More particularly, it deals with the use of sequiterpene lactones (SL), and in particular Helenalin (H. tox) or its derivatives for the treatment of cutaneous or mucosal viral infections, and in particular those associated with warts and other lesions such as those caused by papilloma viruses, herpes simplex viruses and varicella zoster (“chicken pox” and “shingles”).

### B. Related Art

Warts are non-malignant tumors of the epithelium that occur in 13% of individuals in the human population. They are highly prevalent in immunocompromised individuals and can occur in 100% of intentionally immunosuppressed transplant recipients. They are caused by one or more of the 170⁺ strains of the human papilloma virus (HPV). The virus enters the skin through an abrasion and results in the thickening of the stratum corneum. Warts appear in all shapes and sizes and are highly contagions. While warts can appear at any age, they are more likely to bother children and teenagers. Common non-genital types include vulgaris, filiform, plantar and periungal. Nearly all warts are harmless but once they appear on the face, fingers, bottom of the foot, or hands, most people will do almost anything to get rid of them or cover them with cosmetics. They sometimes can become irritated or super-infected by contact with clothing, objects or touching. They are a major cosmetic issue when they appear on the arms, fingers, angles, neck and face. Plantar warts on the bottom of the foot make walking painful.

Without treatment 65% of warts will disappear within months to years, hut 35% remain for decades. Those that do disappear can reappear. Conventional treatments involve freezing, burning, injecting, shaving, electrocuting, laser therapy, poisoning, or digging out the wart, all with varying degrees (30-70%) of success. Treatment of warts can be expensive and involve repeated trips to the dermatologist. In fact, many individuals with warts go to a dermatologist solely for this reason; this population patient represents a large part of most dermatology practices. The treatments themselves can also cause pain, scarring, bleeding, super-infection, oozing and irritation. Nonconventional treatments include everything from duct tape to formaldehyde. Even with aggressive treatment (and often with multiple different treatments) and as noted, warts can re-appear and grow faster, and larger Warts can disappear over a period of months or years, but many remain for a decade or more.

### SUMMARY

Thus, in accordance with the present disclosure, there is provided a method of inhibiting a topical virus infection in a subject in need thereof comprising by topically administering to the subject a composition comprising a sequiterpene lactone (SL) at greater than about 0.0001% w/w and at no more than about 10% w/w to a virally infected area or virally induced lesion. In particular, the SL, Helenalin (H. tox), (±)-4-Hydroxy-4a,8-dimethyl-3,3a,4a,7a,8,9,9a-octahydroazuleno[6,5-b]furan-2,5-dione or its derivatives can be used. The topical composition may comprise H. tox in an amount of about at or up to about 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.01%, 0.005% or 0.001% w/w, or a range of any two of these values. The composition may further comprise an anti-viral or anti-wart agent other than the SL. The composition may be in the form of a cream, a gel, a spray or a salve, a roll-on formulation, or a cosmetic. The SL may be a pharmaceutical ingredient grade SL. The composition may comprise purified SL at about or greater than 0.001% SL, at about or greater than 0.005% SL, at about or greater than 0.01% SL, at about or greater than 0.05% SL, or at about 0.1% SL. The composition may be a homeopathic gel or cream, such as an Arnica Montana or Arnica chamissonis foliosa gel or cream or it might contain purified or synthesized SL such as H. tox.

The virus infection may be caused by a papilloma virus (e.g., HPV), a herpesvirus (HHV) (e.g., herpes simplex 1 virus, herpes simplex 2 virus, varicella zoster virus, HHV-6, HHV-7, varicella zoster virus), hepatitis B virus (HepB) parovirus B19, Epstein Barr virus (EBV), measles virus, or a molluscum contagiosum virus. The HPV may located in a wart or wart-associated skin or mucosal lesion, such as a common wart, filiform wart, genital wart, external genital wart, condyloma acuminata, persistent wart, periungual wart, subungual wart, plantar wart, mosaic wart, recalcitrant wart, palmoplantar wart, flat wart, epidermodysplasia verruciformis related wart, butcher's wart, oropharyngeal wart, laryngeal wart, laryngeal papilloma, laryngeal dysplasia, genital wart, anogenital wart, cervical dysplasia, cervical wart, cervical neoplasia, or a combination thereof.

Administering the composition may further comprise one or more methods that mechanically, physically or chemically enhance penetration of SL into the virally infected area or virally induced lesion or that otherwise may enhance therapeutic efficacy of the composition, such as where mechanically enhancing the penetration of the composition into the virally infected area or virally induced lesion comprises sanding, filing, curetting, or debriding a treatment site of the subject, or such as where mechanically enhancing the penetration of the SL into the virally infected area or virally induced lesion may occur before, after or concurrent with administration of the composition.

Administering the SL composition may comprise once daily, twice daily, weekly, twice weekly, every other week, every other day, monthly, every two months, every three months or as directed by a packaging or a physician to achieve the desired clinical result. Administering the SL composition may continue for at least about one week, at least about two weeks, at least about three weeks, at least about four weeks, at least about five weeks, at least about six weeks, at least about seven weeks, at least about eight weeks, at least about nine weeks, at least about ten weeks, at least about eleven weeks, at least about twelve weeks, at least about one month, at least about two months, at least about three months, at least about four months, at least about five months, at least about six months, a range of any two of these values, or until the virus is inhibited or the lesion is resolved.

Administering the composition may comprise contacting a treatment said virally infected area or virally induced lesion with an applicator selected from the group consisting of a sponge, a swab, a foam tipped stick, a cotton ball, a brush, a woven or non-woven fabric, roller, gauze, a glove, a pen-type applicator, a flocked, doe-foot applicator, or a combination thereof. A feature of the applicator may be used to sand, file, curette, or debride a treatment site of the subject before administration of the SL composition. The method may further comprise covering the virally infected area or virally induced lesion following application of the composition, such as with a bandage, gauze, or film. Administering the composition may comprise contacting the virally infected area or virally induced lesion with a dermal or transdermal patch comprising the composition.

The method may further comprise administering an additional treatment modality comprising a second SL-containing composition, an adjuvant, an inhibitor, tape-stripping, cryotherapy, electrosurgery, curettage, laser therapy, salicylic acid, trichloroacetic acid, podophyllin, cantharidin, 5-fluorouracil, bleomycin, topical imiquimod, intralesional Candida antigen, topical squaric acid dibutyl ester, oral cimetidine, surgical lesion removal, electrodesiccation, lasers of various wavelengths (ablative and non-ablative), radio-frequency ablation, dermabrasion, curettage, surgical excision, an ablative, a chemical peeling agent, disturbing the surface of the lesion, decreasing the thickness or size or overall volume of the lesion, increasing the surface area of the lesion, or a combination thereof.

The wart or virus-associated skin or mucosal lesion may have previously received treatment or have not previously received treatment. The wart may be a recurrent wart or a wart-associated skin or mucosal lesion. The wart or wart-associated skin or mucosal lesion may have persisted in situ for 6 months, 12 months, 2 years, 3 years, 4 years, 5 years or longer. The wart or wart-associated skin or mucosal lesion may be reduced in depth by 50% or more after three weeks of daily treatment, may be reduced in depth by 75% or more after three weeks of daily treatment, may be reduced in depth by 90% or more after three weeks of daily treatment, may be reduced in depth by 50% or more after six weeks of daily treatment, may be reduced in depth by 75% or more after six weeks of daily treatment, may be reduced in depth by 90% or more after six weeks of daily treatment, may be reduced in volume by 50% or more after three weeks of daily treatment, may be reduced in volume by 75% or more after three weeks of daily treatment, may be reduced in volume by 90% or more after three weeks of daily treatment, may be reduced in volume by 50% or more after six weeks of daily treatment, may be reduced in volume by 75% or more after six weeks of daily treatment, may be reduced in volume by 90% or more after six weeks of daily treatment, may remain reduced in diameter or volume by at least 75% for at least three months following treatment, may remain reduced in diameter or volume by at least 75% for at least six months following treatment, or may remain reduced in diameter or volume by at least 75% for at least one year following treatment.

Also provided is kit containing the necessary materials and formulation to treat a wart or wart-associated skin or mucosal lesion. This would comprise a container with a preparation comprising greater than 0.0001% w/w to about 10% w/w of a SL, such as Helenalin (H. tox) (±)-4-Hydroxy-4a,8-dimethyl-3,3a,4a,7a,8,9,9a-octahydroazuleno[6,5-b]furan-2,5-dione or its derivatives. The kit may further comprise one or more of an applicator, a lesion covering, such as a bandage, gauze or film, and more particularly a dermal or transdermal patch, a device for modifying a treatment site to improve absorption of said SL, or an anti-viral or anti-wart agent other than the SL. The SL in the kit may be in the form of a homeopathic gel or cream, such as an Arnica Montana or Arnica chamissonis foliosa gel or cream.

Yet another embodiment comprises a dermal or mucosal covering impregnated with a SL, such as H. tox or its derivatives. The covering may be a patch, gauze or bandage. The patch may be a dermal or transdermal patch. The covering may further comprise an anti-viral or anti-wart agent other than SL. The SL in the covering may be in the form of a homeopathic gel or cream, such as an Arnica Montana or Arnica chamissonis foliosa gel or cream.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed.

FIG. 1. Ankle wart of 35-year-old male pre-treatment.

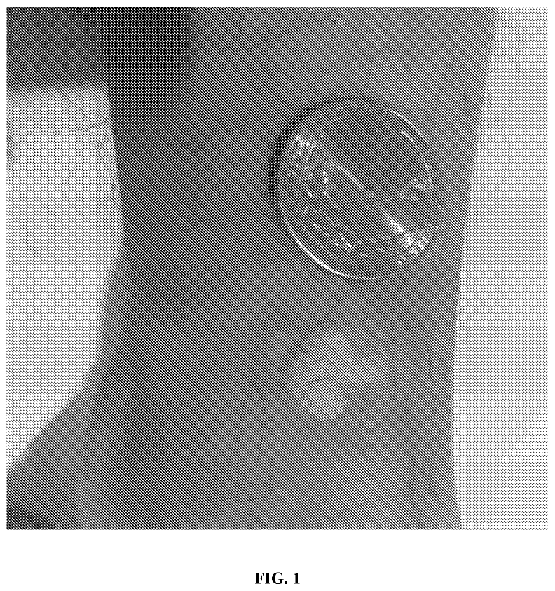

FIG. 2. Ankle wart from FIG. 1 after 17 days of treatment (see Example 1 for treatment protocol).

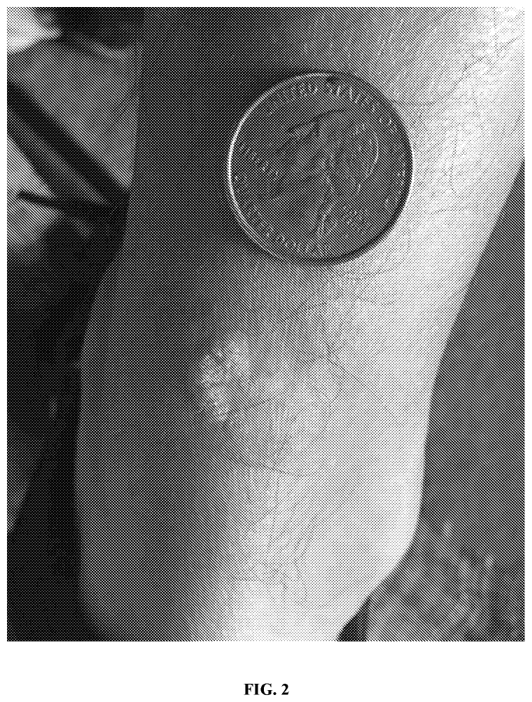

FIG. 3. Ankle wart from FIG. 1 after 43 days of treatment (treatment ceased at this point).

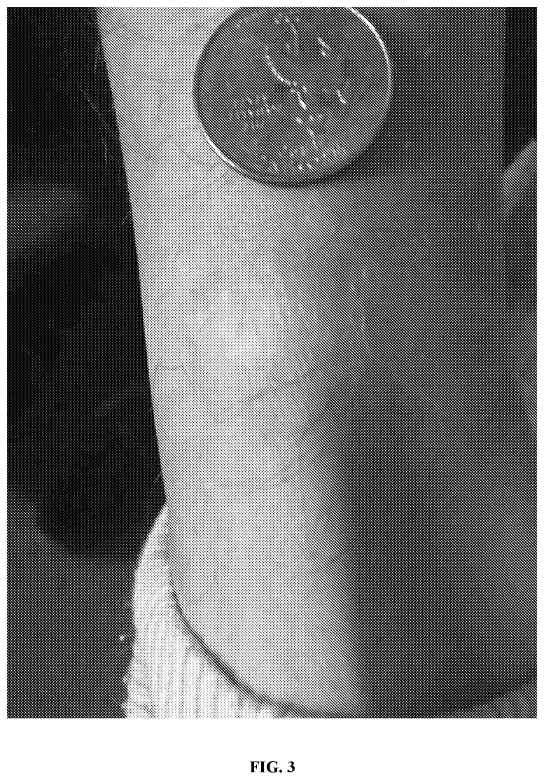

FIG. 4. Ankle wart from FIG. 1 77 days after initial treatment.

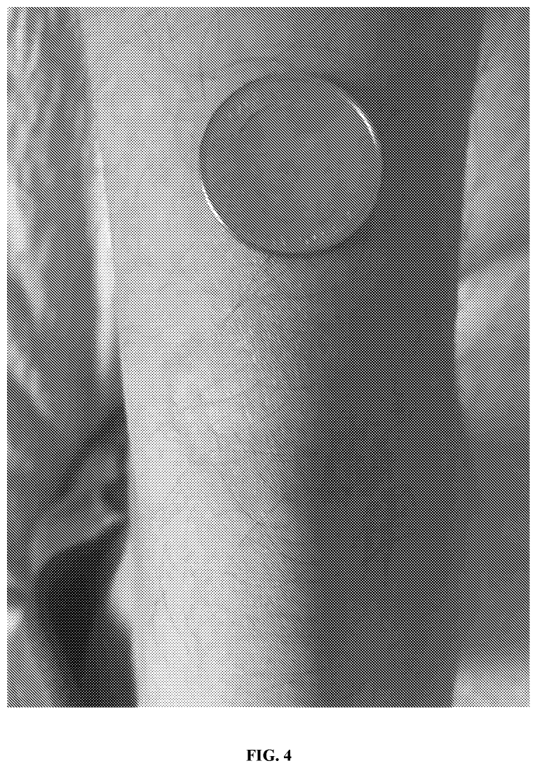

### DETAILED DESCRIPTION

As discussed above, certain warts present a significant treatment challenge, and while not life-threatening, they can be painful and hinder activity as well as damage a person's self-image. As discuss herein, the inventor has demonstrated an effective approach for eliminating warts by simple, painless, non-scaring, and cost-effective topical application. This should be applicable to all warts (including genital warts) since the therapy targets a common molecular target, namely, NF-kappa B (NF-kB) (which is required for the growth of HPV). As such, the inventor posits that this approach should also be effective at treating other NF-kappa B-dependent virally related skin lesions such as The virus infection may be caused by a Herpes Simplex virus-1 (HSV-1), Herpes Simplex virus-2 (HSV-2), varicella zoster virus (chicken pox and herpes zoster, or “shingles”) HepB, parovirus B19, roseola, EBV, rubella/measles, or molluscum contagiosum viruses.

The active ingredients are SLs, including the toxin, H. tox. SLs such as H. tox are found in the flowers of the Arnica Montana and Arnica chamissonis foliosa plants. Like many protein and non-protein toxins produced by plants, e.g., ricin, abrin, gelanin, (pokeweed anti-viral protein (PAP), and ribosome inactivating proteins (RIPs), SLs target transcription and translational of proteins in cells. Applied topically in the commercial formulations (i.e., at very low concentrations), SLs are highly effective and without side effects although hypersensitivities (i.e., allergic reactions such as contact dermatitis) are always possible in a few individuals. H. tox, one of the SLs, has also been used as a highly purified compound and is sold by six companies. It has been shown to inactivate NF-kB by a unique mechanism and is postulated by the inventor to work both an anti-viral and an anti-inflammatory. The combination of these effects may be the key to eliminating cutaneous warts and other local viral lesions. To date the current formulation has been tested on a large ankle wart, a facial wart, and in numerous finger warts in 5 individuals. It has also been tested in a prodrome of oral herpes. All warts have disappeared within 3-5 weeks and have not re-appeared in months (see Example). The prodrome of oral herpes is stopped within 24 hours. These and other aspects of the disclosure are set out in detail below.

### I. Definitions

“Administering”, when used in conjunction with a therapeutic, means to administer a therapeutic directly into or onto a target tissue or to administer a therapeutic to a subject, whereby the therapeutic positively impacts the tissue to which it is targeted. Thus, as used herein, the term “administering”, when used in conjunction with a therapeutic, can include, but is not limited to, providing a therapeutic to a subject systemically by, for example, intravenous injection, whereby the therapeutic reaches the target tissue. Administering a composition or therapeutic may be accomplished by, for example, injection, oral administration, topical administration, or by these methods in combination with other known techniques. Such combination techniques may include heating, radiation, ultrasound and the use of delivery agents. Administering may be a self-administration, wherein the therapeutic or composition is administered by the subject themselves. Alternatively, administering may be administration to the subject by a health care provider.

“Providing”, when used in conjunction with a therapeutic, means to administer a therapeutic directly into or onto a target tissue, or to administer a therapeutic to a subject whereby the therapeutic positively impacts the tissue to which it is targeted.

The term “animal” as used herein includes, but is not limited to, humans and non-human vertebrates such as wild, domestic and farm animals.

The term “patient” or “subject” as used herein is an animal, particularly a human, suffering from an unwanted disease or condition that may be treated by the therapeutic and/or compositions described herein.

The term “improves” is used to convey that the embodiments provided herein change either the characteristics and/or the physical attributes of the tissue to which the therapeutic composition is being provided, applied or administered. The term “improves” may also be used in conjunction with a diseased state such that when a diseased state is “improved” the symptoms or physical characteristics associated with the diseased state are diminished, reduced or eliminated.

The term “inhibiting” generally refers to prevention of the onset of the symptoms, alleviating the symptoms, or eliminating the disease, condition or disorder. “Optional” or “optionally” means that the subsequently described event or circumstance may or may not occur, and that the description includes instances where the event occurs and instances where it does not.

As used herein, “room temperature” means an indoor temperature of from about 20° C. to about 25° C. (68 to 77° F.).

The term “purified H. tox” means an H. tox preparation that contains a greater percentage of H. tox as compared to a natural source of H. tox, such as in Arnica. It would include derivatives of H. tox as well. In certain embodiments, purified H. tox means at least 50%, at least 60%, at least 70%, at least 80%, at least 90%, at least 95%, at least 98%, or at least 99% H. tox, including ranges of 50-99% and 95-88% H. tox.

Throughout the specification of the application, various terms are used such as “primary,” “secondary,” “first,” “second.” and the like. These terms are words of convenience in order to distinguish between different elements, and such terms are not intended to be limiting as to how the different elements may be utilized.

By “pharmaceutically acceptable,” “physiologically tolerable,” and grammatical variations thereof, as they refer to compositions, carriers, diluents, and reagents or other ingredients of the formulation, can be used interchangeably and represent that the materials are capable of being administered without the production of undesirable physiological effects such as rash, burning, irritation or other deleterious effects to such a degree as to be intolerable to the recipient thereof.

As used herein, the term “cosmetically acceptable” and grammatical variations thereof, as they refer to compositions, carriers, diluents, and reagents or other ingredients of the formulation, represent that the materials used and final composition are not irritating or otherwise harmful to the patient in general and to the skin, in particular, and are pleasant and well tolerated with respect to general appearance, pH, color, smell and texture (feel), that they are not, for example, unacceptably sticky (tacky), oily or drying, and that they do spread easily, absorb into the skin at an acceptable rate of absorption, and are generally moisturizing.

As used herein, the term “therapeutic” means an agent utilized to treat, combat, ameliorate, prevent or improve an unwanted condition or disease in a subject. In part, embodiments described herein may be directed to the treatment of various skin diseases, conditions or disorders or symptoms thereof, including, but not limited to, benign proliferations, neoplasms, pre-malignancies or malignancies of the skin. The condition may be a wart-associated condition. The condition may be a virally induced or non-virally induced cutaneous growth or proliferation. The condition may be selected from warts, common warts (Verruca vulgaris), condyloma acuminata, genital warts, external genital warts, palmar and plantar warts, mosaic warts, flat warts, filiform warts butcher's warts, oropharyngeal warts, anogenital warts, laryngeal warts, papilloma, dysplasias (e.g., CIN (cervical intraepithelial neoplasia), persistent warts, periungual warts, subungual warts, recalcitrant warts, treatment naive warts, skin lesions seen in epidermodysplasia verruciformis, molluscum contagiosum, or a combination thereof.

As used herein, the term “stabilized helenalin” or H. tox refers to a H. tox comprising a stabilizer or a blend of stabilizers useful for dilution of the H. tox into a concentration that can be incorporated into a stable commercial formulation for topical application to skin lesions for the treatment of skin conditions described herein. In some embodiments, the H. tox may be obtained from a commercial source. The amount and type of stabilizer(s) used in the H. tox formulation may be proprietary to and/or a trade secret of the commercial source. In some embodiments, the stabilized H. tox is a H. tox of higher concentration than provided in a commercial source. H. tox formulations are typically stable for years at room temperature. Stabilizers may be used in H. tox formulations, usually when obtained through commercial sources, in order to stabilize diluted versions of the “high concentration” H. tox formulation.

As used herein, the term “clinical efficacy” refers to the ability of an ingredient or composition to produce a desired effect. For example, in some embodiments, the desired effect may include, without limitation, decreasing the surface tension of the composition, increasing the wettability of the surface of the skin or skin lesion, increasing the permeability of the composition into the subject's skin, skin lesion, or surface imperfections, including crevices, invaginations, bumps and irregularities of the skin or skin lesion, decreasing the size of the target lesion, improving the shape and/or appearance of the target lesion, improving the target lesion or treated area and/or removing the target lesion, or a combination thereof.

The terms “therapeutically effective” or “effective”, as used herein, may be used interchangeably and refer to an amount of a therapeutic composition of embodiments described herein. For example, a therapeutically effective amount of a composition is an amount of the composition, and particularly the active ingredient, such as H. tox that generally achieves the desired effect.

A “therapeutically effective amount” or “effective amount” of a composition is an amount necessary or enough to achieve the desired result. The activity contemplated by the embodiments herein includes medically therapeutic, cosmetically therapeutic and in some cases prophylactic treatment, as appropriate. The specific dose of a compound administered according to embodiments of the present disclosure to obtain therapeutic and/or prophylactic effects will, of course, be determined by the particular circumstance related to the case, including, for example, the compound administered, the route of administration, and the condition being treated. However, the effective amount administered can be determined by the practitioner or manufacturer or patient in light of the relevant circumstances including the condition to be treated, the choice of compound to be administered, and the chosen route of administration, and therefore, the disclosed dosage ranges are not intended to limit the scope of the disclosure in any way. A therapeutically effective amount of the compound of embodiments herein is typically an amount such that when it is administered in a physiologically tolerable excipient composition, it is enough to achieve an effective systemic concentration or local concentration in or on the tissue to achieve the desired therapeutic or clinical outcome.

The term “tissue” refers to any aggregation of similarly specialized cells which are united in the performance of a function.

The terms “treat,” “treated,” or “treating” as used herein refers to therapeutic treatment, cosmetic treatment and/or prophylactic or preventative measures, wherein the object is to prevent or slow down (lessen) an undesired physiological condition, disorder or disease, or to obtain beneficial or desired clinical results. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects.

As used herein, the term “consists of” or “consisting of” means that the formulation or method includes only the elements, steps, or ingredients specifically recited in the claimed embodiment or claim.

As used herein, the term “consisting essentially of” or “consists essentially of” means that the formulation or method includes only the specified materials or steps and those that do not materially affect the basic and novel characteristics of the claimed disclosure. An example would be the inclusion of non-active ingredients or excipients in a pharmaceutical composition.

### II. Warts

Warts are typically small, rough, and hard growths that are similar in color to the rest of the skin. Some warts have a cauliflower appearance or bumps. They typically do not result in symptoms except when on the bottom of the feet where they may be painful. While they usually occur on the hands and feet, they can also affect other locations. One or many warts may appear. They are not cancerous.

Warts are caused by infection with several different strains the HPV. Factors that increase the risk include use of public showers, sexual contact, exposure to body fluids, working with meat, eczema, and a suppressed immune system. The virus is believed to enter the body through damaged skin. Several types of warts exist including common warts, plantar warts, filiform warts, and genital warts. Immunity to one strain of warts does not guarantee immunity to other strains.

Without treatment, most types of warts resolve in months to years. Several treatments such as salicylic acid applied to the skin and cryotherapy can be effective. These are the most common treatments used by dermatologists. In those who are otherwise healthy these treatments do not typically result in problems other than local burning, oozing, scabbing and scaring. They do require multiple trips to the dermatologists and relapses do occur. Treatment of genital warts differs from that of other types.

Warts are very common, with most people being infected at some point in their life. The estimated current rate of non-genital warts among the general population is 1-13%. They are more common among young people. Estimated rates of genital warts in sexually active women is 12%. Warts have been described at least as far back as 400 BC by Hippocrates.

A range of types of wart have been identified, varying in shape and site affected, as well as the type of HPV involved. These include:
Common wart (Verruca vulgaris), a raised wart with roughened surface, most common on hands, but can grow anywhere on the body. Sometimes known as a Palmer wart or Junior wart.
Flat wart (Verruca plana), a small, smooth flattened wart, flesh-colored, which can occur in large numbers; most common on the face, neck, hands, wrists and knees.
Filiform or digitate wart, a thread- or finger-like wart, most common on the face, especially near the eyelids and lips.
Genital wart (venereal wart, Condyloma acuminatum, Verruca acuminata), a wart that occurs on the genitalia.
Mosaic wart, a group of tightly clustered plantar-type warts, commonly on the hands or soles of the feet.
Periungual wart, a cauliflower-like cluster of warts that occurs around the nails or on the ankles.
Plantar wart (Verruca, Verruca plantaris), a hard sometimes painful lump, often with multiple black specks in the center; usually only found on pressure points on the soles of the feet.

A. Cause

Warts are caused by HPV. There are currently about 170⁺ strains of HPV that have been identified. HPV infects the squamous epithelium, usually of the skin or genitals, but each HPV type is typically thought to only able to infect a few specific areas on the body. Many HPV types can produce a benign growth, often called a “wart” or “papilloma”, in the area they infect. Many of the more common HPV and wart types are listed below and all depend upon NF-kB for growth.
Common warts—HPV types 2 and 4 (most common); also types 1, 3, 26, 29, and 57 and others.
Cancers and genital dysplasia—“high-risk” HPV types are associated with cancers, notably cervical cancer, and can also cause some vulvar, vaginal, penile, anal and some oropharyngeal cancers. “Low-risk” types are associated with warts or other conditions.
High-risk—HPV 16, 18 (cause the most cervical cancer); also 31, 33, 35, 39, 45, 52, 58, 59, and others.
Plantar warts (myrmecia)—HPV type 1 (most common); also types 2, 3, 4, 27, 28, and 58 and others.
Anogenital warts (condylomata acuminata or venereal warts)—HPV types 6 and 11 (most common); also types 42, 44 and others.
Low-risk—HPV 6, 11 (most common); also 13, 44, 40, 43, 42, 54, 61, 72, 81, 89, and others.
Flat warts—HPV types 3, 10, and 28.
Butcher's warts—HPV type 7.
Heck's disease (Focal epithelial hyperplasia)—HPV types 13 and 32.

B. Pathophysiology

Common warts have a characteristic appearance under the microscope. They have thickening of the stratum corneum (hyperkeratosis), thickening of the stratum spinosum (acanthosis), thickening of the stratum granulosum, rete ridge elongation, and large blood vessels at the dermo-epidermal junction.

C. Prevention

Gardasil 6 is an HPV vaccine aimed at preventing cervical cancers and genital warts. Gardasil is designed to prevent infection with HPV types 16, 18, 6, and 11. HPV types 16 and 18 currently cause about 70% of cervical cancer cases, and also cause some vulvar, vaginal, penile and anal cancers. HPV types 6 and 11 are responsible for 90% of documented cases of genital warts. Gardasil 9, approved in 2014 protects against HPV types 6, 11, 16, 18, 31, 33, 45, 52, and 58. HPV vaccines do not currently protect against the virus strains responsible for plantar warts (verrucas). However, it is currently unclear whether it will protect against other types of warts.

D. Disinfection

The virus is relatively hardy and immune to many common disinfectants. Exposure to 90% ethanol for at least 1 minute, 2% glutaraldehyde, 30% Savlon, and/or 1% sodium hypochlorite can disinfect the pathogen. The virus is resistant to drying and heat but killed by 100° C. (212° F.) and ultraviolet radiation.

E. Treatment

There are many treatments and procedures associated with wart removal, although many are only partially effective, particularly with warts that are of significant size and duration. A review of clinical trials of various cutaneous wart treatments concluded that topical treatments containing salicylic acid were more effective than placebo. Cryotherapy appears to be as effective as salicylic acid, but there have been fewer trials. Neither therapy cures warts in all individuals. Below is a more detailed discussion of various treatments.

Salicylic acid can be prescribed by a dermatologist in a higher concentration than that found in over-the-counter products. Several over-the-counter products are readily available at pharmacies and supermarkets of roughly two types: adhesive pads treated with salicylic acid and bottled concentrated salicylic acid solution.

Imiquimod is a topical cream that helps the body's immune system fight the wart virus by encouraging interferon production. It has been approved by the U.S. Food and Drug Administration (FDA) for genital warts.

Cantharidin is found naturally in the bodies of many members of the beetle family Meloidae and causes dermal blistering. It is used either by itself or compounded with podophyllin. Not FDA approved, but available through Canada or select US compounding pharmacies.

Bleomycin is not US FDA approved and can cause necrosis of digits and Raynaud syndrome. The usual treatment is one or two injections.

Dinitrochlorobenzene (DNCB), like salicylic acid, is applied directly to the wart. Studies show this method is effective with a cure rate of 80%. But DNCB must be used much more cautiously than salicylic acid; the chemical is known to cause genetic mutations, so it must be administered by a physician. This drug induces an allergic immune response resulting in inflammation that wards off the wart-causing virus.

Cidofovir is an antiviral drug which is injected into HPV lesions within the larynx (laryngeal papillomatosis) as an experimental treatment.

Benzoyl peroxide (BPO) is effective in the treatment of flat warts.

Silver nitrate, available over the counter, can aid in wart removal in the form of a caustic pencil, which is also available at drug stores. In a placebo-controlled study of 70 patients, silver nitrate given over nine days resulted in clearance of all warts in 43% and improvement in warts in 26% one month after treatment compared to 11% and 14%, respectively, in the placebo group. The instructions must be followed to minimize staining of skin and clothing. Occasionally pigmented scars may develop.

Zinc sulfate, given orally, has been shown to often reduce or eliminate warts. The zinc sulfate dosage used in medical trials for treatment of warts was between 5 and 10 mg/kg/day. For elemental zinc, a lower dosage of 2.5 mg/kg/day may be appropriate as large amounts of zinc may cause a copper deficiency. Other trials have found that topical zinc sulfate solution or zinc oxide are also effective.

Lopinavir has been demonstrated as effective against HPV. The study used the equivalent of one tablet twice a day applied topically to the cervices of women with high-grade and low-grade precancerous conditions. After three months of treatment, 82.6% of the women who had high-grade disease had normal cervical conditions, confirmed by smears and biopsies.

Keratolysis of dead surface skin cells usually using salicylic acid, blistering agents, immune system modifiers (“immunomodulators”), or formaldehyde, often with mechanical paring of the wart with a pumice stone, blade etc.

Electrodessication is a procedure that uses a round dull instrument (curette) of varying sizes (1 mm to 6 mm) is used to scrape off the growth down to the dermis. The scraping is then paused while an electrosurgical device like a hyfrecator is used next. Electrocoagulation (electrodesiccation) is performed over the raw surgical ulcer to denature a layer of the dermis and the curette is used again over the surgical ulcer to remove denatured dermis down to living tissue. In the case of skin cancers, the cautery and electrodesiccation is usually performed three times, or until the surgeon is comfortable that reasonable margins have been achieved.

Cryosurgery or cryotherapy involves freezing the wart (generally with liquid nitrogen), creating a blister between the wart and epidermal layer after which the wart and the surrounding dead skin fall off. An average of 3 to 4 treatments are required for warts on thin skin. Warts on calloused skin like plantar warts might take dozens or more treatments.

Surgical curettage of the wart is also employed. Curettage is the use of a curette (French, meaning scoop) to remove tissue by scraping or scooping

Laser treatment is often performed with a pulse dye laser or carbon dioxide (CO₂) laser. Pulse dye lasers (wavelength 582 nm) work by selective absorption by blood cells (specifically hemoglobin). CO₂ lasers work by selective absorption by water molecules. Pulse dye lasers are less destructive and more likely to heal without scarring. CO₂ laser works by vaporizing and destroying tissue and skin. Laser treatments can be painful, expensive (though covered by many insurance plans), and not extensively scarring when used appropriately. CO₂ lasers will require local anesthetic. Pulse dye laser treatment does not need conscious sedation or local anesthetic. It takes 2 to 4 treatments but can be many more for extreme cases. Typically, 10-14 days are required between treatments. Preventative measures are important.

Infrared coagulator is an intense source of infrared light in a small beam like a laser. This works essentially on the same principle as laser treatment. It is less expensive. Like the laser, it can cause blistering pain and scarring.

Duct tape occlusion therapy involves placing a piece of duct tape over the wart. The mechanism of action of this technique remains unknown. Despite several clinical trials, evidence for the efficacy of duct tape therapy is inconclusive. Despite the mixed evidence for efficacy, the simplicity of the method and its limited side-effects leads some researchers to be reluctant to dismiss it.

### III. Viruses

A. HPV

Verrucae (Warts) are virally induced lesions caused by subtypes of the Cutaneous HPV family. HPV types are a subset of the large group of the DNA papilloma virus family that can infect humans and cause cutaneous lesions. HPV's are ubiquitous in the environment and infection occurs most commonly as a result of direct contact with individuals who harbor the virus either clinically (evidence lesions) or sub-clinically, indirectly through exposure to contaminated surfaces, or by autoinoculation of virus from individual lesions to adjacent uninfected skin. Cutaneous manifestations of HPV infection include common warts (Verruca vulgaris), palmar and plantar warts, mosaic warts, flat warts, butcher's warts, and others. Subtypes of the HPV family are also etiologic of oropharyngeal, anogenital, laryngeal warts, papillomas, dysplasias (e.g., CIN (cervical intraepithelial neoplasia) carcinoma in situ, carcinomas, and in the skin, lesions seen in epidermodysplasia verruciformis. Common warts are typically hyperkeratotic, exophytic dome-shaped papules or nodules (typically associated with HPV types 1, 2 or 4) and are most commonly located on the fingers (including periungual and subungual regions), dorsal surfaces of the hands, sites prone to trauma (e.g., knees, elbows), but may occur at virtually any other anatomical location.

Condyloma acuminata, more commonly known as genital warts, are typically related to HPV types 6 and 11, 16, 18, and numerous other subtypes (e.g., 33, 35, 39, 40, 43, 45, 51-56, 58 and others), and multiple subtypes may exist in a single lesion. Condyloma acuminata typically present as solitary to multiple fleshy, soft, verrucoid papules that may be dome-shaped, filiform, fungating, “cauliflower-like,” or form confluent plaques. They are typically located in the anogenital region (e.g., penis, vulva, vagina, cervix, perineum, and perianal regions), and may appear in the oropharynx, larynx and even tracheal mucosa, and rarely other cutaneous locations (e.g., trunk, extremities). The lesions are typically benign, but certain HPV subtypes are associated with a risk of malignant potential (e.g., HPV subtypes 16, 18) and may lead to cutaneous carcinomas or carcinomas-in-situ such as bowenoid papulosis or Buschke-Lowenstein tumor, and cervical dysplasias or neoplasia, e.g., cervical intraepithelial neoplasia (CIN).

In immunocompetent individuals, many common cutaneous lesions associated with HPV infection (e.g., warts and condylomata) appear and some spontaneously resolve in less than two years. However, warts can be large and/or cosmetically unsightly (e.g., face, hands), spread to distant anatomical regions by autoinoculation, painful (e.g., traumatized or on soles of feet), and untreated warts provide a significant reservoir of HPV infection in the community.

B. Mollusca

Virally induced lesions can be caused by subtypes of the DNA poxvirus family of molluscum contagiosum viruses (MCV). There are four subtypes of MCV, (MCV-1 to 4), with MCV-1 being the most prevalent and MCV-2 being most common in adults. Like HPV's, MCV's are ubiquitous in the environment and infection occurs most commonly as a result of direct contact with individuals who harbor the virus either clinically (evidence lesions) or sub-clinically, indirectly through exposure to contaminated surfaces, or by autoinoculation of virus from individual lesions to adjacent uninfected skin. The infection is most common in the pediatric population, sexually active adults, and the immunocompromised. Molluscum contagiosum lesions are typically flesh-colored, dome-shaped umbilicated (“dimpled”) papules that may occur singly or in clusters and are typically located on the trunk groin or extremities, though may occur on any area of the skin. Individual lesions may spontaneously resolve in several weeks to several months, however, the natural history of the infection from appearance of the first lesions to resolution of the last lesion may last from six months to five years or more.

There are currently no U.S. Food and Drug Administration approved treatments for molluscum contagiosum. There are currently no specific antiviral therapies available to treat cutaneous MCV infection. Existing therapies are thus directed towards either the direct physical destruction of the lesions with locally destructive modalities such as cryotherapy, electrosurgery, curettage, laser therapy, unroofing the lesion with e.g., a needle (“needle-pricking”), application of acids or caustics (e.g., salicylic acid, potassium hydroxide); locally cytotoxic therapies, such as topical podophyllin, cantharidin; topical immunomodulatory therapy (e.g., topical imiquimod, intralesional Candida antigen, nitric acid, oral cimetidine) or surgical lesion removal. While these methods can achieve cure rates in some cases, many require multiple visits to a physician's office, specialized training and the use of expensive equipment; they may be painful and may require anesthesia and/or analgesia, and may be anxiety producing and psychologically traumatic, particularly in the pediatric age group. These treatments may be complicated by adverse cosmetic outcomes including pigment changes (both hyperpigmentation and hypopigmentation), scarring at the treatment site, bleeding and infection. No one therapy is consistently effective in all cases and in fact, there is great variability among practitioners in the methods employed using each of these techniques with great variability of the results. The use of multiple treatment modalities including the treatment of underlying topical conditions such as atopic dermatitis, which tends to predispose to molluscum and the spread of the lesions, in combination is often necessary to achieve significant improvement. Thus, there exists a great unmet need in the art for a safe and efficacious (topical) treatment for the cutaneous lesions associated with MCV infection.

C. Herpesviruses

Herpesviridae, encompassing the herpes viruses, is a large family of DNA viruses that cause diseases in animals, including humans. The members of this family are also known as herpesviruses. The family name is derived from the Greek word herpein (“to creep”), referring to the latent, recurring infections typical of this group of viruses. Herpesviridae can cause latent or lytic infections. In total, there are more than 130 herpes viruses, some of them from mammals, birds, fish, reptiles, amphibians, and mollusks.

At least five species of Herpesviridae—HSV-1 and HSV-2 (both of which can cause oro-labial herpes and genital herpes), varicella zoster virus (the cause of chickenpox), EBV (implicated in several diseases, including mononucleosis and some cancers and often causing a rash), and cytomegalovirus—are extremely widespread among humans. More than 90% of adults have been infected with at least one of these, and a latent form of the virus remains in most people.

There are 9 herpesvirus types known to infect humans: herpes simplex viruses 1 and 2, HSV-1 and HSV-2, (also known as HHV1 and HHV2), varicella-zoster virus (VZV, which may also be called by its ICTV name, HHV-3), EBV or HHV-4, human cytomegalovirus (HCMV or HHV-5), human herpes virus 6A and 6B (HHV-6A and HHV-6B), human herpes virus 7 (HHV-7), and Kaposi's sarcoma-associated herpes virus (KSHV, also known as HHV-8). Most of these viruses are associated with skin lesions or malignancies.

There are antiviral drugs that are effective against some kinds of herpes virus. Herpesviruses all share a common structure; they are composed of relatively large double-stranded, linear DNA genomes encoding 100-200 genes encased within an icosahedral protein cage called the capsid which is itself wrapped in a protein layer called the tegument containing both viral proteins and viral mRNAs and a lipid bilayer membrane called the envelope. This whole particle is known as a virion.

Reactivation of latent viruses has been implicated in a number of diseases (e.g., Shingles, Pityriasis Rosea, measles). Following activation, transcription of viral genes transitions from latency-associated LAT to multiple lytic genes; these lead to enhanced replication and virus production. Often, lytic activation leads to cell death. Clinically, lytic activation is often accompanied by emergence of non-specific symptoms such as low grade fever, headache, sore throat, malaise, and rash as well as clinical signs such as swollen or tender lymph nodes and immunological findings such as reduced levels of natural killer cells.

D. Hep B Virus

Hepatitis B is caused by Hep-B virus, a hepadnavirus that can cause both acute and chronic hepatitis. Chronic hepatitis develops in the 15% of adults who are unable to eliminate the virus after an initial infection. Identified methods of transmission include blood (blood transfusion, now rare), unsanitary tattoos, sexually (through sexual intercourse or through contact with blood or bodily fluids), or via mother to child by breast feeding (minimal evidence of trans-placental crossing). However, in about half of cases the source of infection cannot be determined. Blood contact can occur by sharing syringes in intravenous drug use, shaving accessories such as razor blades, or touching wounds on infected persons. Needle-exchange programs have been created in many countries as a form of prevention.

Patients with chronic Hep-B have antibodies against the virus, but these antibodies are often insufficient to clear the infection of the affected liver cells. The continued production of virus combined with antibodies is a likely cause of the immune complex disease seen in these patients. A vaccine is available that will prevent infection from Hep-B for life. Hep B infections result in 500,000 to 1,200,000 deaths per year worldwide due to the complications of chronic hepatitis, cirrhosis, and hepatocellular carcinoma. Hep-B is endemic in number of (mainly South-East Asian) countries, making cirrhosis and hepatocellular carcinoma big killers. There are six treatment options approved by the U.S. Food and Drug Administration (FDA) available for persons with a chronic Hep-B infection: alpha-interferon, pegylated interferon, adefovir, entecavir, telbivudine, and lamivudine. About 65% of persons on treatment achieve a sustained response.

One symptom of HBV infection is the appearance of skin lesions. (Prior to the appearance of skin lesions there is usually fever and a prodome that involves tingling in the area where the blister will appear.) The most common are essential mixed cryoglobulinemia and vasculitis, followed by papular changes and purpuric and maculopapular rash. Petechia and purpura also have been reported.

E. Parvovirus

Primate erythroparvovirus 1, generally referred to as B19 virus, parvovirus B19 or sometimes erythrovirus B19, was the first (and until 2005 the only) known human virus in the family Parvoviridae, genus Erythroparvovirus; it measures only 23-26 nm in diameter. The name is derived from Latin, parvum meaning small, reflecting the fact that B19 ranks among the smallest DNA viruses. B19 virus is most known for causing disease in the pediatric population; however, it can also affect adults. It is the classic cause of the childhood rash called fifth disease or erythema infectiosum, or “slapped cheek syndrome.”

Erythroviruses belong to the Parvoviridae family of small DNA viruses. It is a non-enveloped, icosahedral virus that contains a single-stranded linear DNA genome. The infectious particles may contain either positive or negative strands of DNA. The icosahedral capsid consists of two structural proteins, VP1 (83 kDa) and VP2 (58 kDa), which are identical except for 227 amino acids at the amino-terminal of the VP1-protein, the so-called VP1-unique region. Each capsid consists of a total of 60 capsomers: VP2 is the major capsid protein and comprises approximately 95% of the total virus particle. VP1-proteins are incorporated into the capsid structure in a non-stochiometrical relation (based on antibody-binding analysis and X-ray structural analysis the VP1-unique region is assumed to be exposed at the surface of the virus particle. At each end of the DNA molecule there are palindromic sequences which form “hairpin” loops. The hairpin at the 3′ end serves as a primer for the DNA polymerase. It is classified as erythrovirus because of its capability to invade red blood cell precursors in the bone marrow. Three genotypes (with subtypes) have been recognized.

The Nucleotide substitution rate for total coding DNA has been estimated to be 1.03 (0.6-1.27)×10⁻⁴ substitutions/site/year. This rate is similar to that of other single stranded DNA viruses. VP2 codons were found to be under purifying selection. In contrast VP1 codons in the unique part of the gene were found to be under diversifying selection. This diversifying selection is consistent with persistent infection as this part of the VP1 protein contains epitopes recognized by the immune system.

Like other non-enveloped DNA viruses, pathogenicity of parvovirus B19 involves binding to host cell receptors, internalization, translocation of the genome to the host nucleus, DNA replication, RNA transcription, assembly of capsids and packaging of the genome, and finally cell lysis with release of the mature virions. In humans the P antigen (also known as globoside) is the cellular receptor for parvovirus B19 virus that causes Erythema infectiosum (fifth disease) in children. This infection is sometimes complicated by severe aplastic anemia caused by lysis of early erythroid precursors.

The virus is primarily spread by infected respiratory droplets; blood-borne transmission, however, has been reported. The secondary attack risk for exposed household persons is about 50%, and about half of that for classroom contacts.

Symptoms begin some six days after exposure (between 4 and 28 days, with the average being 16 to 17 days) and last about a week. Infected patients with normal immune systems are contagious before becoming symptomatic, but probably not after then. Individuals with B19 IgG antibodies are generally considered immune to recurrent infection, but reinfection is possible in a minority of cases. About half of adults are B19-immune due to a past infection.

A significant increase in the number of cases is seen every three to four years; the last epidemic year was 1998. Outbreaks can arise especially in nurseries and schools. Parvovirus B19 causes an infection only in human. Parvoviruses from cats and dogs do not infect humans. There is no vaccine available for human parvovirus B19, though attempts have been made to develop one.

Fifth disease or erythema infectiosum is only one of several expressions of Parvovirus B19. The associated bright red rash of the cheeks gives it the nickname “slapped cheek syndrome.” Individuals can be affected at any age, although it is most common in children aged six to ten years. It is so named because it was the fifth most common cause of a pink-red infection associated rash to be described by physicians (many of the others, such as measles and rubella, are rare now).

Once infected, patients usually develop the illness after an incubation period of four to fourteen days. The disease commences with high fever and malaise, when the virus is most abundant in the bloodstream, and patients are usually no longer infectious once the characteristic rash of this disease has appeared. The following symptoms are characteristic:
a usual brief viral prodrome with fever, headache, nausea, diarrhea;
as the fever breaks, a red rash forms on the cheeks, with relative pallor around the mouth (“slapped cheek rash”), sparing the nasolabial folds, forehead, and mouth;
“lace-like, (reticular)” red rash on trunk or extremities then follows the facial rash. Infection in adults usually only involves the reticular rash, with multiple joint pain predominating;
exacerbation of rash by sunlight, heat, stress;
teenagers or young adults may develop the so-called “Papular Purpuric Gloves and Socks Syndrome”;
“slapped cheek” appearance typical of fifth disease.
   Other presentations include chronic anemia in individuals who have AIDS, arthritis and arthralgias in adults, aplastic crisis, which is most dangerous in patients with pre-existing bone marrow stress, hydrops fetalis

Currently, there are no treatments that directly target Parvovirus B19 virus. Intravenous immunoglobulin therapy (IVIG) therapy has been a popular alternative because doctors can administer it without stopping chemotherapy drugs like MEL-ASCT. Also, the treatment's side effects are rare as only 4 out of 133 patients had complications (2 had acute renal failure and 2 had pulmonary edema) even though 69 of the patients had organ transplants and 39 of them were HIV positive. However, it is important to note that IVIG therapy is not perfect as it is required forever, and even then, 34% of treated patients will have a relapse after 4 months even with continued treatment.

F. Rubella

Rubella virus (RuV) is the pathogenic agent of the disease rubella and is the cause of congenital rubella syndrome when infection occurs during the first weeks of pregnancy. RuV v is the only member of the genus Rubivirus and belongs to the family of Togaviridae, whose members commonly have a genome of single-stranded RNA of positive polarity which is enclosed by an icosahedral capsid.

Rubella, also known as German measles or three-day measles, is the infection caused by the rubella virus. This disease is often mild with half of people not realizing that they are infected. A rash may start around two weeks after exposure and last for three days. It usually starts on the face and spreads to the rest of the body. The rash is sometimes itchy and is not as bright as that of measles. Swollen lymph nodes are common and may last a few weeks. A fever, sore throat, and fatigue may also occur. In adults joint pain is common. Complications may include bleeding problems, testicular swelling, and inflammation of nerves. Infection during early pregnancy may result in a child born with congenital rubella syndrome (CRS) or miscarriage. Symptoms of CRS include problems with the eyes such as cataracts, ears such as deafness, heart, and brain. Problems are rare after the 20th week of pregnancy.

Rubella is usually spread through the air via coughing. People are infectious during the week before and after the appearance of the rash. Babies with CRS may spread the virus for more than a year. Only humans are infected. Insects do not spread the disease. Once recovered, people are immune to future infections. Testing is available that can verify immunity. Diagnosis is confirmed by finding the virus in the blood, throat, or urine. Testing the blood for antibodies may also be useful.

Rubella is preventable with the rubella vaccine with a single dose being more than 95% effective. Often it is given in combination with the measles vaccine and mumps vaccine, known as the MMR vaccine. When some, but less than 80% of the people are vaccinated, more women might make it to childbearing age without developing immunity by infection or vaccination and CRS rates could increase. Once infected there is no specific treatment.

Rubella is a common infection in many areas of the world. Each year about 100,000 cases of congenital rubella syndrome occur. Rates of disease have decreased in many areas as a result of vaccination. There are ongoing efforts to eliminate the disease globally. In April 2015 the World Health Organization declared the Americas free of rubella transmission.

Rubella has symptoms that are like those of flu. However, the primary symptom of rubella virus infection is the appearance of a rash (exanthem) on the face which spreads to the trunk and limbs and usually fades after three days (that is why it is often referred to as three-day measles). The facial rash usually clears as it spreads to other parts of the body. Other symptoms include low grade fever, swollen glands (sub-occipital and posterior cervical lymphadenopathy), joint. When the rash clears up, the skin might shed in very small flakes where the rash covered it. Forchheimer's sign occurs in 20% of cases, and is characterized by small, red papules on the area of the soft palate.

Rubella can affect anyone of any age and is generally a mild disease, rare in infants or those over the age of 40. The older the person is the more severe the symptoms are likely to be. Up to 60% of older girls or women experience joint pain or arthritic type symptoms with rubella.

Rubella infections are prevented by active immunization programs using live attenuated virus vaccines. Two live attenuated virus vaccines, RA 27/3 and Cendehill strains, were effective in the prevention of adult disease. However, their use in pre-pubertal females did not produce a significant fall in the overall incidence rate of CRS in the UK. Reductions were only achieved by immunization of all children.

The vaccine is now usually given as part of the MMR vaccine. The WHO recommends the first dose be given at 12 to 18 months of age with a second dose at 36 months. Pregnant women are usually tested for immunity to rubella early on. Women found to be susceptible are not vaccinated until after the baby is born because the vaccine contains live virus.

There is no specific treatment for rubella; however, management is a matter of responding to symptoms to diminish discomfort. Treatment of newborn babies is focused on management of the complications. Congenital heart defects and cataracts can be corrected by direct surgery.

Management for ocular congenital rubella syndrome (CRS) is similar to that for age-related macular degeneration, including counseling, regular monitoring, and the provision of low vision devices, if required.

G. Roseola

Roseola is an infectious disease caused by certain herpes viruses, which are discussed above. Most infections occur before the age of three. Symptoms vary from absent to the classic presentation of a fever of rapid onset followed by a rash. The fever generally lasts for three to five days. The rash is generally pink and lasts for less than three days. Complications may include febrile seizures, with serious complications being rare.

It is caused by either human herpesvirus 6 (HHV-6) or human herpesvirus 7 (HHV-7), which are sometimes referred to collectively as Roseolovirus. There are two variants of HHV-6. (HHV-6a and HHV-6b) and studies in the US, Europe, Dubai and Japan have shown that exanthema subitum is caused by HHV-6b. This form of HHV-6 infects over 90% of infants by age 2. Spread is usually through the saliva of those who are otherwise healthy. However, it may also spread from the mother to baby during pregnancy. Diagnosis is typically based on symptoms but can be confirmed with blood tests. Low numbers of white blood cells may also be present.

Treatment includes sufficient fluids and medications to treat the fever. Nearly all people are infected at some point in time. Males and females are affected equally often. The disease was first described in 1910 while the causal virus was determined in 1988. The disease may reactivate in those with a weakened immune system and may result in significant health problems. Most cases of HHV-6 infection get better on their own. If encephalitis occurs ganciclovir or foscarnet may be useful.

Roseola typically affects children between six months and two years of age and begins with a sudden high fever (39-40° C.; 102.2-104° F.). In rare cases, this can cause febrile convulsions (also known as febrile seizures or “fever fits”) due to the sudden rise in body temperature, but in many cases the child appears normal. After a few days the fever subsides, and just as the child appears to be recovering, a red rash appears. This usually begins on the trunk and then spreads to the arms, legs, and neck. The rash is not itchy and may last 1 to 2 days. In contrast, a child suffering from measles would usually appear sicker, with symptoms of conjunctivitis, cold-like symptoms, and a cough, and their rash would affect the face and last for several days. Liver dysfunction can occur in rare cases.

A small percentage of children acquire HHV-6 with few signs or symptoms of the disease. Exanthema subitum occurs in approximately 30% of children during primary HHV-6 infection. Others may show symptoms significant enough that other more serious infections, such as meningitis or measles should be ruled out. In case of febrile seizures, medical advice can be sought for reassurance. However, febrile seizures are not harmful, do not require treatment, and have no long-term negative effects unless they last longer than five minutes.

In rare cases, HHV-6 can become active in an adult previously infected during childhood and can show signs of mononucleosis.

### IV. H. tox and Compositions Containing the Same

A. H. tox

H. tox, or (±)-4-Hydroxy-4a,8-dimethyl-3,3a,4a,7a,8,9,9a-octahydroazuleno[6,5-b]furan-2,5-dione, is a toxic sesquiterpene lactone which can be found in several plants such as Arnica Montana and Arnica chamissonis subsp. foliosa. Although toxic, H. tox has some in vitro anti-inflammatory and anti-neoplastic effects at non-toxic concentrations. It can inhibit certain enzymes, such as 5-lipoxygenase and leukotriene C4 synthase. For this reason, the compound or its derivatives may have potential medical applications.

Structure and reactivity. H. tox belongs to the group of SLs that are characterized by a lactone ring. The structures of several compounds in this group are shown below:

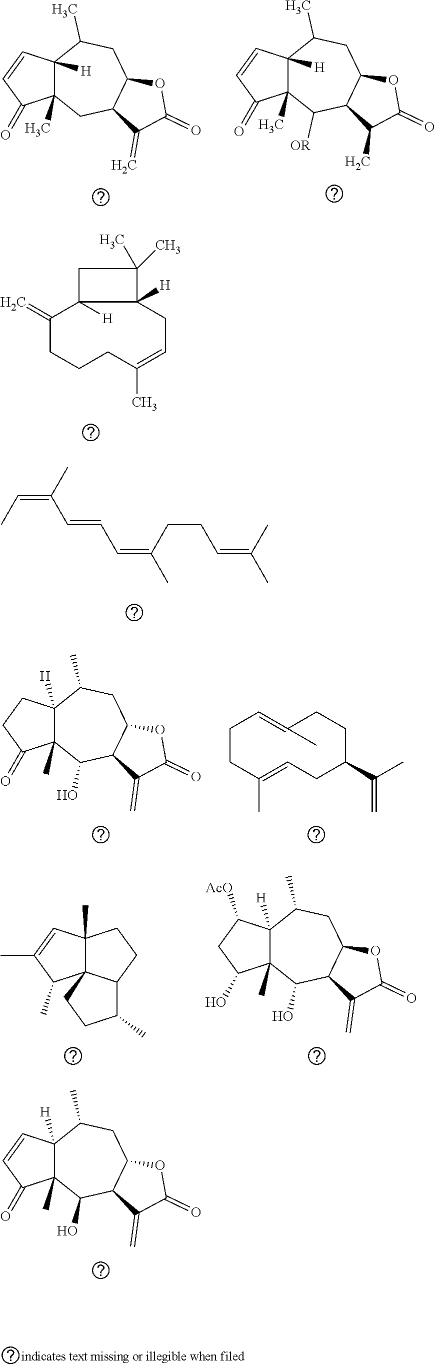

Beside the ring structure, H. tox has two reactive groups (a-methylene-γ-butyrolactone and a cyclopentenone group) that can undergo a Michael addition. The double bond in the carbonyl group can undergo a Michael addition with a thiol group, also called a sulfhydryl group. Therefore, H. tox can interact with proteins by forming covalent bonds to the thiol groups of cysteine-containing proteins/peptides, such as glutathione. This effect can disrupt the molecule's biological function. Addition reactions can occur because thiol groups are strong nucleophiles; a thiol has a lone pair of electrons.

Other SLs related to H. tox are found within the same SL family, the pseudoguaianolides. Most of these derivatives occur naturally, such as the compound dihydrohelenalin, but there are also some semi-synthetic derivatives known, such as 2β-(S-glutathionyl)-2,3-dihydrohelenalin. In general, most derivatives are more toxic than H. tox itself. Among these, derivatives with the shortest ester groups are most likely to contain a higher toxicity. Other derivatives include 11a,13-dihydrohelenalin acetate, 2,3-dehydrohelenalin and 6-O-isobutyrylhelenalin. The molecular conformation differs between H. tox and its derivatives, which affects the lipophilicity and the accessibility of the Micheal addition sites. Poorer accessibility results in a compound with lower toxicity. Another possibility is that a derivative lacking one of the reactive groups, such as the cyclopentenone group, may have a lower toxicity.

Biochemical effects of H. tox. H. tox targets the p65 subunit (also called RelA) of the transcription factor NF-κB. This transcription factor is required for the synthesis of many pro-inflammatory cytokines as well as the transcription of several viruses within cells. It can react with Cys38 in RelA by Michael addition. Both reactive groups, α-methylene-γ-butyrolactone and cyclopentene, can react with this cysteine. It was also found that H. tox can inhibit human telomerase, a ribonucleoprotein complex, by Michael addition. In this case also, both reactive groups of H. tox can interact with the thiol group of a cysteine and inhibit the telomerase activity. H. tox inhibits the formation of leukotrienes in human blood cells by inhibiting LTC4 synthase activity. H. tox reacts with its cyclopentenone ring to the thiol group of the synthase.

Metabolism. H. tox inhibits cytochrome P450 enzymes by reacting with thiol groups, resulting in inhibition of the mixed-function oxidase system. These effects are important for the cytotoxicity of H. tox. The levels of glutathione, which contains sulfhydryl groups, are reduced in H. tox-treated cells, further increasing the toxicity of H. tox. Depending on the dose of H. tox, thiol-bearing compounds such as glutathione may provide some protection to cells from H. tox toxicity. It was also seen that H. tox increase CPK and LDH activities in serum and that it inhibits multiple enzymes of the liver involved in triglyceride synthesis. Therefore H. tox causes acute liver toxicity, accompanied by a decrease cholesterol levels. Chapman et al., Fundamental Applied Toxicology, 10(2): 302-312, 1988. H. tox also suppresses essential immune functions, such as those mediated by activated CD4⁺ T-cells, by multiple mechanisms. Berges et al., Mol. Immunol., 46(15): 2892-2901, 2009.

In vitro anti-inflammatory and anti-neoplastic effects. H. tox and some of its derivatives have been shown to have potent anti-inflammatory and anti-neoplastic effects in vitro. Some studies have suggested that the inhibition by H. tox of platelet leukotriene C4 synthase, telomerase activity and transcription factor NF-kB contribute to H. tox's in vitro anti-inflammatory and anti-neoplastic activity. The dose used varied per study. There is currently no in vivo evidence regarding H. tox's anti-inflammatory and anti-tumor effects, if any.

Effect of H. tox on cells in culture. The inventor has compared the toxicity of highly purified H. tox on HeLa cells and Jurkat cells following 24-48 hrs. in culture. The positive control was ricin and the negative control, vehicle only. While ricin has an expected IC₅₀ of 10⁻¹² to 10⁻¹³M, H. tox had an IC₅₀ of approximately 10⁻⁶ M. Hence, H. tox is 1-10 million-fold less toxic to cells than ricin. It's IC₅₀ over a 24-48 hr. period is consistent with its inhibition of NF-kB.

Application. Historically, plant extracts containing H. tox have been used as an herbal medicine for the treatment sprains, bruises, blood clots, muscle strain and rheumatic complaints. Currently H. tox is used topically in homeopathic gels and microemulsions. H. tox is not FDA-approved for medical application.

Toxicity. When applied topically on humans, H. tox can cause contact dermatitis in a small number of sensitive individuals. However, it is considered safe when applied this way. In individuals with a history of contact dermatitis, the topical preparation can be used in a pre-treatment test application of two applications as week apart. Oral administration of large doses of H. tox can cause gastroenteritis, muscle paralysis, cardiac and liver damage. The toxicity of H. tox was studied in mammalian species such as mice, rat, rabbit and sheep, were the oral LD₅₀ of H. tox was established between 85-150 mg/kg. It was shown in a mouse model that H. tox caused reduces levels of cholesterol. In a rat model, alcohol hepatic injury was prevented by H. tox administration. Parenteral administration showed a higher toxic effect when compared to oral administration.

Pharmacology. H. tox has a variety of observed effects in vitro including anti-inflammatory and anti-tumor activities. H. tox has been shown to selectively inhibit the transcription factor NF-KB, which plays a key role in regulating immune responses, through a unique mechanism. In vitro, it is also a potent, selective inhibitor of human telomerase—which may partially account for its antitumor effect. It also has anti-trypanosomal activity and is toxic to Plasmodium falciparum.

Animal and in vitro studies have also suggested that H. tox can reduce the growth of Staphylococcus aureus and reduce the severity of S. aureus infection. Hence, it might be useful in treating skin lesions (boils, rashes, pustules) caused by Staph.

B. Sources of H. tox

Arnica Montana. One source of H. tox is the ethnobotanical Arnica montana. A. montana, also known as wolf s bane, leopard's bane, mountain tobacco and mountain Arnica. The names “wolfs bane” and “leopard's bane” are also used for another plant, aconitum, which is extremely poisonous. It is used as an herbal medicine for analgesic and anti-inflammatory purposes. Clinical trials have produced mixed results.

The main constituents of Arnica Montana are essential oils, fatty acids, thymol, pseudoguaianolid, SLs and flavanone glycosides. Pseudoguaianolide sesquiterpenes constitute 0.2-0.8% of the flower head of Arnica Montana. H. tox fatty esters 2,5-Dimethoxy-p-cymene and thymol methyl ether are the primary components of essential oils from both the plant's roots and rhizomes.

Although Arnica Montana's main pharmacologically active constituent H. tox is safe and beneficial in the very small quantities used in herbal medicine (as an anti-inflammatory and analgesic), it is extremely toxic and almost always fatal in larger doses. Using whole plant material can result in sudden death, and only standardized preparations of the plant should be used (sparingly) for medicinal purposes (even then, concentrated, purified, and standardized pharmaceutical-grade preparations of H. tox are considered highly preferable due to their significantly more accurate dosing and lack of pharmaceutically active minor Arica Montana alkaloids which are invariably present in plant-derived extracts). Many of the plant's trace-alkaloids are more toxic than H. tox by one or more orders of magnitude, acting as potent hepatotoxins, cytotoxins, mutagens, teratogens, and neurotoxic central nervous system stimulants.

Two techniques for preparing Arnica Montana extracts are hydroalcoholic maceration and gentle disintegration in soybean oil. Propylene glycol and butylene glycol extractions were also reported. The composition of these extracts can include fatty acids, especially palmitic, linoleic, myristic, and linolenic acids, essential oil, triterpenic alcohols, sesquiterpene lactones, sugars, phytosterols, phenol acids, tannins, choline, inulin, phulin, arnicin, flavonoids, carotenoids, coumarins, and heavy metals.

No consensus exists on topical dosing for Arnica. Experiments show absorption of active chemical constituents to be dependent on the concentration and form of preparation. Microemulsion preparations exhibit greater absorption, as do more concentrated forms of the tincture. Creams typically contain 15% Arnica oil and salves have 20% to 25% of Arnica oil.

Arnica chamissonis. Another source of H. tox is Arnica chamissonis, the Chamisso arnica, is a North American species of plants in the sunflower family. It is very similar to Arnica Montana. Arnica chamissonis is native to the United States and Canada and naturalized in parts of Europe while A. Montana is indigenous to Europe.

C. Pharmaceutical Compositions

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render materials stable and facilitate for interaction with target cells or lesions. Aqueous compositions of the present disclosure comprise an effective amount of an SL, dissolved or dispersed in a pharmaceutically acceptable carrier (ethanol or DMSO) and diluted into aqueous medium. Such compositions also are referred to as inoculum. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the compositions of the present disclosure, its use in therapeutic compositions is contemplated. Supplementary active ingredients also can be incorporated into the compositions.

Embodiments in this disclosure are directed to a pharmaceutical composition comprising SLs such as H. tox. In some embodiments, the composition may further include an alcohol or DMSO. In some embodiments, the SL may be stabilized SL. In some embodiments, the SL may be a standard grade, food grade, chemical synthesis grade, drinking water grade, pharmaceutical grade, active pharmaceutical ingredient (API) grade, or cosmetic grade SL. In some embodiments, the topical composition comprises 10% or up to about 10% w/w of the composition, such as at or up to about 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.01%, 0.005% or 0.001% w/w.

In some embodiments, the alcohol may be selected from a primary alcohol, a secondary alcohol, a tertiary alcohol, or a combination thereof. In some embodiments, the alcohol may be selected from, but is not limited to, a low molecular weight alcohol, such as methanol, ethanol, butanol, 1-propanol, pentanol, hexanol, octanol, nonanol, decanol, 2-butanol, 2-propanol, 2-pentanol, benzyl alcohol, an isomer thereof, or a combination thereof. In some embodiments, the alcohol is not 1-propanol, ethanol, propanol, butanol, pentanol, hexanol, octanol, nonanol, decanol, 2-butanol, 2-pentanol, or benzyl alcohol. In some embodiments, the alcohol is 2-propanol (also referred to as isopropyl alcohol). In some embodiments, other volatiles such as, for example, acetates such as ethyl and butyl acetate (volatiles used in nail lacquers), cyclomethycaine (a volatile silicone which may be included in an emulsifier system) may be used in combination with or in place of an alcohol. Embodiments herein also include a composition consisting essentially of an SL and an alcohol. Some embodiments are directed to a composition consisting of an SL and an alcohol. Some embodiments are directed to a composition comprising an SL and 2-propanol. Some embodiments are directed to a composition consisting essentially of an SL and 2-propanol. Some embodiments are directed to a composition consisting of an SL and 2-propanol.

In some embodiments, the composition may comprise an agent that decreases the surface tension of the composition, such as an alcohol. In some embodiments, the surface tension modifying agent may be in an amount sufficient to decrease the surface tension of the composition to a level that effectively increases the penetration of the composition into such crevices and/or invaginations of the skin lesion, increase the surface area of reaction, increase the therapeutic efficacy and/or clinical response of the skin lesion to the therapeutic composition while minimizing irritation of the surrounding skin, or any combination thereof. Furthermore, alcohols that lower the surface tension of an SL composition excessively may run the risk of easily spreading off the application site and across non-lesional skin, causing less activity at the needed site and unwanted irritation and other adverse effects on the surrounding, unaffected skin. In some embodiments, the alcohol is in an amount up to about 5% w/w of the composition.

In some embodiments, the composition may be formulated for topical administration. In some embodiments, the composition may be a solution. In some embodiments, the composition may be in a gel formulation. In some embodiments, the solution or gel formulation may be in two or more parts to be admixed at or immediately before the time of administration. In some embodiments, the composition may be in a cream, lotion, ointment, foam, roll-on formulation, transdermal patch, powder, solid, tape, paste or tincture. In some embodiments, the methods of treating described in embodiments herein require only one single application of the composition of embodiments herein. In some embodiments, the methods of treating described in embodiments herein require two or more applications of the composition of embodiments herein. In some embodiments, the methods of treating described in embodiments herein require multiple applications of the composition of embodiments herein. In some embodiments, the treated area should be covered by a bandage or tape.

In some embodiments, the composition may further include a pharmaceutically acceptable excipient. In some embodiments, the composition may further include an emollient, an emulsifier, a gelling agent, an additive, or a combination thereof. In some embodiments, the additive may be selected from preservatives, emulsion stabilizers, pH adjusters, chelating agents, viscosity modifiers, antioxidants, surfactants, detergents, emollients, opacifying agents, skin conditioners, buffers, or a combination thereof.

In another embodiment, the composition is a cosmetic composition, and a topical cosmetic composition. The composition may comprise a preservative, including but not limited to a pigment or other coloring agent, sodium benzoate, benzoic acid, sorbic acid, benzethonium chloride, benzalkonium chloride, bronopol, methylparaben, ethylparaben, propylparaben, butylparaben, thiomerosol, sodium propionate, chlorhexidine, chlorobutanol, chlorocresol, cresol, imidazolidinyl urea, diazolidinyl urea, phenol, phenylmercuric salts, potassium sorbate, propylene glycol, or mixtures thereof. In certain embodiments, the composition further comprises a permeation enhancer, such as dimethyl isosorbide, isopropyl myristate, diethylene glycol monoethyl ether (e.g., TRANSCUTOL® HP brand diethylene glycol monomethyl ether), ethyl alcohol, ethyl oleate, isostearyl alcohol, oleyl alcohol, polyethylene glycol, N-methyl-2-pyrrolidone, dimethyl sulfoxide, or propylene carbonate. The cosmetic formulation may be oil-in-water emulsion, or a water-in-oil emulsion. The oil phase may comprise cod liver oil, light mineral oil, heavy mineral oil, shark liver oil, caprylic/capric triglyceride, vegetable oil, or mixtures thereof. In certain embodiments, the oil phase comprises a biocompatible oil, such as triacetin, diacetin, tocopherol, or mineral oil. Other biocompatible oils that may be used include such oils listed in U.S. Pat. No. 5,633,226 (incorporated in its entirety by reference herein) and include CAPTEX®. 200, WHITEPSOL®. H-15 and MYVACET® 9-45K, hydrogenated cocoa oil, coconut oil, elm seed oil, palm oil, cottonseed oil, soybean oil, parsley seed oil, mustard seed oil, linseed oil, Tung oil, pomegranate seed oil, laurel oil, rapeseed oil, corn oil, evening primrose oil, maize oil, olive oil, persic oil, poppy-seed oil, safflower oil, sesame oil, soya oil, sunflower oil, ethyl oleate oil, Japanese anise oil, oil of eucalyptus, rose oil, almond oil, arachis oil, castor oil, mineral oil, peanut oil, vegetable oil and derivatives, sucrose polyester, silicone oil, and paraffin oil. The vegetable oil, for example, may comprise castor oil, corn oil, canola oil, cottonseed oil, peanut oil, sesame oil, or soybean oil. The cosmetic formulation may be a lotion, a cream, a gel, or a gel-cream. In other embodiments, the formulation is in the form of ointments; emulsions, preferably in the form of creams, milks or pomades; powders, impregnated pads or adherent applicators such as patches, solutions, gels, sprays, lotions, suspensions, soaps, and shampoos. They may also be in the form of suspensions of microspheres or nanospheres or of lipid or polymer vesicles or of polymer patches and/or of hydrogels allowing controlled release. These compositions may be in anhydrous form, in aqueous form or in the form of an emulsion.

The formulations of the disclosure can be varied in terms of the potency and dosage, or in terms of the ingredients. All requirements for manufacturing the other embodiments of the formulations of the disclosure are the same as described for the formulation.

Gels are preferred vehicles of dispensing formulations of the disclosure. Gels are more accurately called jellies because they are compounds of water-soluble ingredients, usually clear, of a uniform semisolid consistency. Gels maintain a uniformity that is useful in keeping active ingredients in the formulations evenly dispersed in their aqueous base. Brownian motion builds up networks in gels and restores their shape when they have been ruptured by stresses (e.g., settling or shaking). The longer the dosage contacts the skin the longer the duration of action. As a formulation of the disclosure is in a gel base that soothes the skin without a socially objectionable odor. One is less likely to want to wash away the homeopathic formulation from the skin.

In addition to the gel base, various other gel base formulations can carry the active ingredients in the homeopathic formulations of the disclosure with varying degrees of success. However, the use of glycerin and calendula (1X) and other homeopathics in a water gel base of the formulation is very emollient, non-drying, and soothing to the skin. Other carrier bases may also be used to deliver the active ingredients topically such as: water, alcohol, water/alcohol, cream ointment, salves, lotion, liniment, tinctures, cream gel, lotion ointment, rub, spray, aerosol, lotion spray, balm rub, gel ointment, lotion cream, poultice, plaster, infusion, decoction and other herbal methods of preparation. However, the gel delivery system does work best.

In some embodiments, the gel formulation may further comprise an alcohol. In some embodiments, the gel formulation may further comprise a pharmaceutically acceptable excipient. In some embodiments, the gelling agent may be selected from Carbopol ETD 2020, Carbopol 980 NF, Carbopol 974P, Carbopol Ultrez 10, or the like. In some embodiments, the gelling agent may be high molecular weight, cross linked copolymers of acrylic acid and a hydrophobic comonomer or a copolymer (e.g., Pemulen TR-1); Polycarbophil AA-1; PVP (polyvinyl pyrrolidone); Eudragit; Poloxamer; Sepineo; Bentonite; Aerosil (silicates); Hyaluronic Acids; cross-linked Hyaluronic Acids; a combination thereof, or a compositional or chemical equivalent thereof possessive of the qualities required for the composition of embodiments described herein. In some embodiments, the gel composition is kept in two-parts and mixed at or immediately before the time of application. For example, the SL and 2-propanol (first part) could be kept separate from the gelling agent (second part) until the time of administration or immediately before. As another example, the SL, 2-propanol and gelling agent could each be separated into three parts and mixed at or immediately before the time of application. Additional parts may be possible for additional excipients or such excipients may be incorporated into existing parts. At or immediately before the time of administration, the multi-part gel formulation may be mixed and applied topically to the skin as a single gel formulation.

In addition to the active ingredients described above, the formulations of the disclosure optionally further contain one or more physiologically acceptable carriers and/or excipients. An example of the physiologically acceptable carriers and/or excipients is purified, such as in sterile, water. The formulations of the disclosure may be made in a clear gel base. The clear gel base may comprise water, glycerin (e.g., vegetable glycerin USP), a polyacrylic acid resin thickener (e.g., CARBOPOL® 940), triethanolamine and methylparaben.

Some embodiments are directed to a gel formulation that may be delivered in an applicator that mixes two or more components of the gel formulation at or immediately before the time of application. In some embodiments, the gel formulation compartment applicator comprises at least one frangible compartment (e.g., gelling agent in the main compartment of the applicator, with the peroxide in a glass ampule within or alongside that compartment). Some exemplary applicators may include syringe-like applicators or “double-barrel” applicators that can freshly mix (e.g., “vortex mix”) two or more components that need to be held in separate compartments for stability reasons, but which can be mixed at or immediately before the time of application.

In some embodiments, the composition further includes a buffer. In some embodiments, the buffer may be selected from triethanolamine, low pH buffers such as sodium acetate, citrate, phosphate, glycine, hydrogen chloride, citrate and phosphate, glycine and hydrogen chloride, the like, or a combination thereof. In some embodiments, the buffer may be present in an amount of about 0.001% w/w to about 15% w/w. In some embodiments, the buffer is present in an amount of about 0.001% w/w, 0.01% w/w, 0.05% w/w, 0.1% w/w, 0.5% w/w, 1% w/w, 5% w/w, 10% w/w, 15% w/w, or a range of any two of these values. In some embodiments the buffer is present in any amount necessary to optimally adjust the pH of the composition.

In some embodiments, the composition has a pH of about 1.5 to about 7.0. In some embodiments, the pH may be about 1.5 to about 3.5, about 1.5 to about 5.0, about 1.5 to about 4.0, about 1.7 to about 3.7, about 2.0 to about 5.0, about 2.0 to about 4.0, about 2.0 to about 2.8, about 2.5 to about 4.0, about 2.5 to about 4.5, about 2.5 to about 5.0, about 2.7 to about 3.83, about 2.7 to about 4.0, about 2.8 to about 4.0, about 2.83 to about 3.83, about 3.0 to about 7.0, about 4.0 to about 7.0, about 5.0 to about 7.0, or about 6.0 to about 7.0. In some embodiments, the pH may be about 1.5, 1.7, 2.0, 2.5, 2.7, 2.8, 2.83, 3.0, 3.3, 3.5, 3.7, 3.83, 4.0, 4.5, 5.0, 5.5, 6.0, 6.5, 7.0, or a range of any two of these values.

The composition may be stable for at least about two years at 5° C., at least about 12 months at 30° C., at least about 12 months at 25° C., at least about 12 months at 5° C., at least about 6 months at 5° C., at least about 6 months at 25° C., at least about 6 months at 40° C., at least about 3 months at 5° C., at least about 3 months at 25° C., at least about 3 months at 40° C., or a combination thereof.

### V. Methods of Treatment Using Pharmaceutical Formulations of SLs

A. Methods

The methods of the disclosure can be applied to a wide range of species, e.g., humans, non-human primates (e.g., monkeys, baboons, or chimpanzees), horses, cattle, pigs, sheep, goats, dogs, cats, rabbits, guinea pigs, gerbils, hamsters, rats, mice, amphibians and reptiles).

It has been observed that a large, multi-relapsed periungual wart and 3 common wart lesions evidenced a clinical response after the application of the compositions of embodiments herein. In certain cases, improvement, reduction, or clearance of the smaller cutaneous lesions was observed after just several treatments In some embodiments, the warts may be selected from common warts (Verruca vulgaris), condyloma acuminata genital warts, external genital warts, palmar and plantar warts, mosaic warts, flat warts, filiform warts butcher's warts, oropharyngeal warts, anogenital warts, laryngeal warts, persistent warts, periungual warts, subungual warts, recalcitrant warts, treatment naive warts, or a combination thereof. Some embodiments describe a method of treating molluscum contagious. In cases, e.g., with persistent, recurrent, treatment resistant, or with thicker lesions or larger lesions, several weeks of treatment may be required for a clinical response. In certain cases, improvement or clearance of the cutaneous lesions was observed after a series of treatments (e.g. twice daily for several weeks). As exemplary benefits of this treatment method, the clinical response was well-tolerated and was brought about without the need for analgesia, without inducing pain, and without inducing the significant adverse events and adverse cosmetic outcomes commonly resulting from other therapies such as, e.g., pigment changes (such as hypopigmentation or hyperpigmentation), scarring at the treatment site, bleeding or infection. The treatments can be done in minutes.

Embodiments herein are also directed to a method of treating warts or a wart associated condition in a subject in need thereof comprising administering a composition of embodiments herein to a subject in need thereof. In some embodiments, the composition may be administered once daily, twice daily, weekly, twice a week, three times a week, every other day, monthly, every two months, every three months or as directed by the packaging or the physician to achieve the desired clinical result. In some embodiments, the composition is administered once weekly. In some embodiments, the composition is administered twice weekly. In some embodiments, the composition is administered once or at regular intervals for one week, two weeks, three weeks, four weeks, five weeks, six weeks, seven weeks, eight weeks, nine weeks, ten weeks, eleven weeks, twelve weeks, one month, two months, three months, four months, five months, six months, or a range of any two of these values. In some embodiments, the composition is administered for at least about one week, at least about two weeks, at least about three weeks, at least about four weeks, at least about five weeks, at least about six weeks, at least about seven weeks, at least about eight weeks, at least about nine weeks, at least about ten weeks, at least about eleven weeks, at least about twelve weeks, at least about one month, at least about two months, at least about three months, at least about four months, at least about five months, at least about six months, a range of any two of these values, or until the lesion is resolved. In some embodiments, the composition is administered until the lesion is resolved. In some embodiments, the composition may be administered once weekly for eight weeks. In some embodiments, the composition may be administered twice weekly for eight weeks. In some embodiments, the composition may be administered once daily for one week and then administered once weekly or twice weekly until the lesion is resolved. Any other number of combinations are possible and will be readily apparent to one skilled in the art.

In some embodiments, the subject achieves improvement or partial clearance of the skin lesion after treatment with the composition. In some embodiments, the subject achieves complete clearance of the skin lesion during treatment with the composition. In some embodiments, the subject achieves complete clearance of the skin lesion after the course of treatment with the composition. In some embodiments, the lesion continues to improve and/or resolve after the course of treatment. In some embodiments, the subject may achieve improvement in or complete clearance of the condition and/or lesions surrounding or distant to the site of treatment. In some embodiments the lesions might not appear if the viral prodrome is treated.

In some embodiments, the method of treating warts or a wart associated condition further comprises the step of cleansing the treatment site before administration of the composition. In some embodiments cleansing the treatment site comprises applying an alcohol to the skin to be treated before administration of the composition. The treatment may comprise applying the alcohol onto the skin, such as by rubbing, massaging, placing, scrubbing, abrading, wiping, swabbing, or otherwise contacting the skin with the alcohol.

In some embodiments, the method of treating warts or a wart associated condition further comprises the step of debriding the treatment site of the subject. In some embodiments, debriding may include mechanically, chemically, or physically abrading, ablating, thinning, curetting, destroying, removing, excising, or otherwise disturb the surface of the skin or lesion to be treated, including decreasing the thickness of, and/or decreasing the volume of the lesion. In some embodiments, the step of debriding is before, after, and/or concurrent with administration of the compositions of embodiments herein to the treatment site.

In some embodiments, pretreating the skin may comprise applying another known agent to the skin such as ethyl acetate, butyl acetate, or the like onto the skin, such as by rubbing, massaging, placing, scrubbing, abrading, wiping, swabbing, or otherwise contacting the skin with the agent. In some embodiments, cleansing the skin comprises applying an antiseptic solution to the skin or skin lesion to be treated. In some embodiments, the antiseptic is povidone, iodine, chlorhexidine, a detergent, soap or the like.

In some embodiments, warts may be treated using any of the methods of treatment described herein. Some embodiments are directed to a method of improving the appearance of warts comprising administering a composition of embodiments herein to a subject in need thereof. Some embodiments are directed to a method of alleviating, e.g., shrinking, reducing in size, and/or reducing in height, a wart comprising administering a composition of embodiments herein to a subject in need thereof. In some embodiments, the warts are treatment naive. In some embodiments, a treatment naive wart may be treated using any of the methods of treatment described herein. Some embodiments are directed to a method of improving the appearance of treatment naive warts comprising administering a composition of embodiments herein to a subject in need thereof. Some embodiments are directed to a method of improving the appearance, such as by use of a cosmetic concealer comprising the SL, of treatment naive warts comprising administering a composition of embodiments herein to a subject in need thereof. Some embodiments are directed to a method of alleviating, e.g., shrinking, reducing in size, and/or reducing in height, a treatment naive wart comprising administering a composition of embodiments herein to a subject in need thereof. In some embodiments, the warts are not treatment-naive or are recalcitrant warts. Recalcitrant warts may include warts that are resistant to or have failed or have partially responded to other therapies, warts that are difficult to treat, or warts that are in anatomical locations that are difficult to treat or are known to have lower response to treatment or higher recurrence rates. In some embodiments, recalcitrant warts may include plantar warts, periungal warts, subungual warts or the like. In some embodiments, recalcitrant warts may be located on any surface of the body. In some embodiments, recalcitrant warts may be treated using any of the methods of treatment described herein. Some embodiments are directed to a method of improving the appearance of recalcitrant warts comprising administering a composition of embodiments herein to a subject in need thereof. Some embodiments are directed to a method of improving the appearance of recalcitrant warts comprising administering a composition of embodiments herein to a subject in need thereof. Some embodiments are directed to a method of alleviating, e.g., shrinking, reducing in size, and/or reducing in height, a recalcitrant wart comprising administering a composition of embodiments herein to a subject in need thereof. In some embodiments, the subject achieves improvement or partial clearance of the condition after treatment with the composition. In some embodiments, the subject achieves complete clearance of the warts after treatment with the composition. In some embodiments, the subject achieves a Physician Wart Assessment score of clear or mild after treatment with the composition. In some embodiments, the subject achieves a reduction of at least one ordinal on the Physician Wart Assessment score after treatment with the composition. In some embodiments, the subject achieves a reduction of at least two ordinals on the Physician Wart Assessment score after treatment with the composition. In some embodiments, the subject achieves a reduction of at least three ordinals on the Physician Wart Assessment score after treatment with the composition.

Some embodiments are directed to a method of treating a herpes virus-induced lesion, an HBV-induced lesion or prodrome, a parvovirus-induced lesion, a MCV-induced lesion, a poxvirus-induced lesion, a rubella virus-induced lesion, a HPV-induced lesion or other virus-induced lesion comprising administering a composition of embodiments herein to a subject in need thereof. In some embodiments, the subject achieves improvement or partial clearance of the lesion after treatment with the composition. In some embodiments, the subject achieves complete clearance of the lesion after treatment with the composition. In some embodiments, the method of treating comprises administering a composition of embodiments herein to a subject in need thereof, wherein the SL is in an amount of about 0.005% w/w to about 10% w/w.

Some embodiments herein are directed to a method of treating a skin condition comprising (i) topically administering a first dose of a composition of embodiments herein; and (ii) topically administering one or more follow-up doses of the composition. In some embodiments, the application of the first dose and one or more follow-up doses comprises one application session. In some embodiments, the follow-up dose is administered immediately after administration of the first dose. In some embodiments, the follow-up dose is administered at least about 0.5 minutes after the first dose. In some embodiments, the follow-up dose is administered at least about 1 minute, at least about 1.5 minutes, at least about 2 minutes, at least about 5 minutes, at least about 10 minutes, at least about 15 minutes, at least about 20 minutes, at least about 25 minutes, at least about 30 minutes, or at least about 1 hour after the first dose. In some embodiments, there may be one follow-up dose, two follow-up doses, three follow-up doses, four follow-up doses, five follow-up doses, six follow-up doses, seven follow-up doses, eight follow-up doses, nine follow-up doses or ten follow-up doses in each application session. Each subsequent follow-up dose may be administered immediately after administration of the previous dose, at least about 1 minute, at least about 1.5 minutes, at least about 2 minutes, at least about 5 minutes, at least about 10 minutes, at least about 15 minutes, at least about 20 minutes, at least about 25 minutes, at least about 30 minutes or at least one hour after the previous dose. The lesion may be covered once treated with a skin covering, such as a bandage, gauze or film or spray-on composition.

In some embodiments, topical administration of the first dose and the follow-up doses comprises massaging the composition into the skin for at least about 5 seconds, at least about 10 seconds, at least about 15 seconds, at least about 20 seconds, at least about 25 seconds, at least about 30 seconds, at least about 35 seconds, at least about 40 seconds, at least about 45 seconds, at least about 50 seconds, at least about 55 seconds, at least about 1 minute, at least about 2 minutes, at least about 3 minutes, at least about 4 minutes, or at least about 5 minutes, at least about 10 minutes, at least about 15 minutes, at least about 20 minutes at least about 25 minutes, or at least about 30 minutes during each dose. In some embodiments, the composition is a solution. In some embodiments, the composition is a gel. In some embodiments, massaging includes rubbing-in, manipulating, kneading, pressing, or otherwise “working” the composition into the skin. In some embodiments, the composition is applied and massaged into the skin with an applicator. In some embodiments, the composition is applied and massaged into the skin at least 1 time, at least 2 times, at least 3 times, at least 4 times, at least 5 times, at least 6 times, at least 7 times, at least 8 times, at least 9 times, or at least 10 times in each application session. In some embodiments, the application session, in which the composition is administered, may be repeated once daily, twice daily, weekly, biweekly, three times a week, every other day, monthly, every two months, every three months, every six months or as directed by the packaging or the physician to achieve the desired clinical result.

In some embodiments, the SL composition may be administered at least about 1 day, at least about 2 days, at least about 3 days, at least about 1 week, at least about 2 weeks, at least about 3 weeks, at least about 4 weeks, at least about 5 weeks, at least about 6 weeks, at least about 7 weeks, at least about 8 weeks, at least about two months, at least about 3 months, at least about 4 months, at least about 5 months, at least about 6 months, or at least about one year after the initial treatment. In some embodiments, the take home composition may be administered once daily, twice daily, weekly, biweekly, every other day, monthly, or every two months.

In some embodiments, the SL composition may be administered using a sponge, a swab, a foam tipped stick, a cotton ball, a brush, a woven or non-woven fabric, roller, gauze, a pen, a glove, or the like. In some embodiments, the applicator may dispense compositions of embodiments herein via a tip that is flocked, absorbent, and/or firm enough to apply pressure to the lesion. In some embodiments the applicator is a sintered polymeric-tip applicator or a roll-on applicator. In some embodiments, the applicator is resistant to, compatible with, or inert to high concentration peroxide solutions. In some embodiments, the applicator can dispense the solution at a controlled rate in order to help confine the active to the lesion of interest and spare surrounding normal skin. In some embodiments, the solution or gel may be administered using a device having the solution or gel in a compartment that dispenses the solution onto or through an applicator tip when needed. In some embodiments, the applicator may comprise a material designed to abrade the skin lesion or treatment site before, after, or at the time of administration of the composition.

B. Routes of Administration and Delivery Devices

Regarding transdermal delivery, a patch is particularly contemplated. There are five main types of transdermal patches. In the Single-layer Drug-in-Adhesive, the adhesive layer of this system also contains the drug. In this type of patch, the adhesive layer not only serves to adhere the various layers together, along with the entire system to the skin, but is also responsible for the releasing of the drug. The adhesive layer is surrounded by a temporary liner and a backing. In Multi-layer Drug-in-Adhesive, the multi-layer drug-in adhesive patch is similar to the single-layer system in that both adhesive layers are also responsible for the releasing of the drug. One of the layers is for immediate release of the drug and other layer is for control release of drug from the reservoir. The multi-layer system is different however that it adds another layer of drug-in-adhesive, usually separated by a membrane (but not in all cases). This patch also has a temporary liner-layer and a permanent backing.

Unlike the Single-layer and Multi-layer Drug-in-adhesive systems, the reservoir transdermal system has a separate drug layer. The drug layer is a liquid compartment containing a drug solution or suspension separated by the adhesive layer. This patch is also backed by the backing layer. In this type of system, the rate of release is zero order.

The Matrix system has a drug layer of a semisolid matrix containing a drug solution or suspension. The adhesive layer in this patch surrounds the drug layer partially overlaying it. Also known as a monolithic device.

In Vapor Patches, the adhesive layer not only serves to adhere the various layers together but also to release vapor. The vapor patches are new on the market and they release essential oils for up to 6 hours. The vapor patches release essential oils and are used in cases of decongestion mainly, although controller vapor patches that improve the quality of sleep or reduce cigarette usage.

The active compounds may also be administered parenterally or intraperitoneally. Solutions of the active compounds as free base or pharmacologically acceptable salts can be prepared in water suitably mixed with a surfactant, such as hydroxypropyl cellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, using a coating, such as lecithin, by the maintenance of the required particle size in the case of dispersion and using surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the SLs of the present disclosure may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including gels, pastes, powders and slurries. The active ingredient may be added in a therapeutically effective amount to a paste dentifrice that may include water, binders, abrasives, flavoring agents, foaming agents, and humectants.

The compositions of the present disclosure may be formulated in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with enough saline or glucose. These aqueous solutions are especially suitable for intravenous, intramuscular, subcutaneous and intraperitoneal administration. In this connection, sterile aqueous media which can be employed will be known to those of skill in the art considering the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, preparations should meet sterility, pyrogenicity, safety and purity standards as required by FDA Office of Biologics standards.

C. Kits

Some embodiments herein are directed to a kit for treating warts or other viral lesions comprising a container comprising an SL. In some embodiments, the container is a frangible container. In some embodiments, the kit may further include a gel agent or a gelling agent. In some embodiments, the container further includes an applicator. In some embodiments, the SL comprised within an applicator.

### VI. Combination Therapies

Treating viral infections or virally induced lesions may involve the co-administration of a SL and another agent or therapy that also can treat the same. The agents/therapies would be provided in a combined amount effective to treat one or more aspect of the infection or lesion, and/or the underlying physiologic processes that cause it. This process may involve contacting the patient with the agents/therapies at the same time. This may be achieved by contacting the patient with a single composition or pharmacological formulation that includes both agents/therapies, or by contacting the cell with two distinct compositions/treatments or formulations, at the same time, wherein one composition includes an SL and the other includes the other agent or therapy.

Alternatively, the SL treatment may precede or follow the other agent or therapy by intervals ranging from minutes to weeks. In embodiments where the other agent or therapy and SL composition are applied separately to the subject, one would generally ensure that a significant period did not expire between each delivery, such that the agents/therapies would still be able to exert an advantageously combined effect on the subject. In such instances, it is contemplated that one would administer both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either SL composition or the other agent or therapy will be desired. Various combinations may be employed, where SL composition is “A,” and the other agent/therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations, including chronic and continuous dosing of one or both agents/therapies, are contemplated.

Standard agents/therapies for treating viral infections and virally induced lesions, including warts, include methods that mechanically, physically or chemically enhance the penetration of the active into the lesion or that otherwise may enhance the therapeutic efficacy of the composition. Such methods may include tape-stripping, destructive/ablative modalities such as, e.g. cryotherapy, electrosurgery, curettage, laser therapy, application of acids (e.g., salicylic acid, trichloroacetic acid); locally cytotoxic therapies, such as topical podophyllin, cantharidin, or topical or intralesional 5-fluorouracil, or bleomycin; topical immunomodulatory therapy (e.g., topical imiquimod, intralesional Candida antigen, topical squaric acid dibutyl ester, oral cimetidine) or surgical lesion removal, electrodesiccation, lasers of various wavelengths (ablative and non-ablative), radio-frequency ablation, dermabrasion, and partial or complete surgical removal by curettage or surgical excision, use of an ablative or chemical peeling agent, or otherwise disturbing the surface of, or decreasing the thickness or size or overall volume of, or increasing the surface area of the lesion.

Alternatively, the combination therapy may include other active ingredients or agents useful for treating viral infections, viral lesions and/or warts. The agents and treatments are disclosed above and include, but are not limited to, administering salicylic acid to the lesion of a person in need thereof, administering cryotherapy, laser therapy or another locally destructive therapy to a lesion.

### VII. Examples

The following examples are included to demonstrate embodiments of the disclosure. It should be appreciated by those of skill in the art that the techniques disclosed in the examples which follow represent techniques discovered by the inventor to function well in the practice of the disclosure, and thus can be considered to constitute modes for its practice. However, those of skill in the art should, considering the present disclosure, appreciate that many changes can be made in the specific embodiments which are disclosed and still obtain a like or similar result without departing from the spirit and scope of the disclosure.

### Example 1

An otherwise healthy 35-year-old male had a large ankle wart that appeared in 2012. Between 2015 and 2017 he received five courses of deep cryotherapy, resulting in pain, inflammation and scabbing. He has been on 250 mg/day of oral Famciclovir since 2014. The wart regrew after each treatment and eventually became larger. In fact, it was growing on top of the cryotherapy scar. He then used a crushed aspirin under a commercial band aid for a period of several weeks. The wart was slightly reduced in size but when treatment was stopped it regrew. He began treatment with commercially available Helenalin-containing Arnica gel (Borion). This involved putting a small dab on the surface of the wart and covering it with a band aid once in the AM and again in the late PM. There were no noticeable side effects. Within 3 weeks the wart was noticeably smaller. After another 3 weeks (6 weeks total) the wart was gone, and treatment was stopped. After 18 months the wart remains in remission/cure and the scar is fading.

Although not pictured, it should also be noted that the same source of SL, used several times daily as a hand cream for 2-4 weeks, completely cured small finger warts in 5 individuals in the absence of any side effects. One individual had suffered from numerous finger warts for over 20 years and is now wart-free. No side effects or scaring were noted in any of the individuals undergoing treatment for finger warts. It should also be noted that when administered during the prodrome phase of an oral herpesvirus outbreak, the blister never appeared.

All the compositions and/or methods disclosed and claimed herein can be made and executed without undue experimentation considering the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VIII. References

The following references, to the extent that they provide exemplary procedural or other details supplementary to those set forth herein, are specifically incorporated herein by reference:

### Remington's Pharmaceutical Sciences, 15th Edition.

1. A method of inhibiting a topical virus infection in a subject in need thereof comprising topically administering to the subject a composition comprising Helenalin (H. tox; (±)-4-Hydroxy-4a,8-dimethyl-3,3a,4a,7a,8,9,9a-octahydroazuleno[6,5-b]furan-2,5-dione or its derivatives) at greater than about 0.0001% w/w and at no more than about 10% w/w to a virally infected area or virally induced lesion.

2. The method of claim 1, wherein the topical composition comprises H. tox or its derivatives in an amount of about at or up to about 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.01%, 0.005% or 0.001% w/w, or a range of any two of these values.

3. The method of claim 1, wherein the composition further comprises an anti-viral or anti-wart agent other than the H. tox or its derivatives.

4. The method of claim 1, wherein the composition is in the form of a cream, a gel, a spray or a salve.

5. The method of claim 1, wherein said virus infection may be caused by a human papilloma virus, a herpesvirus (e.g., herpes simplex 1 virus, herpes simplex 2 virus, varicella zoster virus, human herpes virus 6, human herpes virus 7, a varicella zoster virus), Hep-B virus, Parvovirus B19, EBV, measles virus, or a molluscum contagiosum virus, such as wherein said HPV is in a wart or wart-associated skin or mucosal lesion.

6-7. (canceled)

8. The method of claim 1, wherein administering the composition further comprises one or more methods that mechanically, physically or chemically enhance penetration of H. tox or its derivatives into the virally infected area or virally induced lesion or that otherwise may enhance therapeutic efficacy of the composition.

9-10. (canceled)

11. The method of claim 1, wherein administering the H. tox composition comprises once daily, twice daily, weekly, twice weekly, every other week, every other day, monthly, every two months, every three months or as directed by a packaging or a physician to achieve the desired clinical result.

12. The method of claim 1, wherein administering the H. tox composition continues for at least about one week, at least about two weeks, at least about three weeks, at least about four weeks, at least about five weeks, at least about six weeks, at least about seven weeks, at least about eight weeks, at least about nine weeks, at least about ten weeks, at least about eleven weeks, at least about twelve weeks, at least about one month, at least about two months, at least about three months, at least about four months, at least about five months, at least about six months, a range of any two of these values, or until the virus is inhibited or the lesion is resolved.

13. The method of claim 1, wherein the H. tox is a pharmaceutical ingredient grade H. tox.

14. The method of claim 1, wherein the composition comprises purified H. tox of greater than 0.005% H. tox, of greater than 0.01% H. tox, or greater than 0.05% H. tox, or greater than 0.1% H. tox.

15. The method of claim 1, wherein administering the composition comprises contacting a treatment said virally infected area or virally induced lesion with an applicator selected from the group consisting of a sponge, a swab, a foam tipped stick, a cotton ball, a brush, a woven or non-woven fabric, roller, gauze, a glove, a pen-type applicator, a flocked, doe-foot applicator, or a combination thereof.

16. (canceled)

17. The method of claim 1, further comprising covering the virally infected area or virally induced lesion following application of the composition, such as with a bandage, gauze, or film.

18. The method of claim 1, wherein administering the composition comprises contacting the virally infected area or virally induced lesion with a dermal or transdermal patch comprising the composition.

19. The method of claim 1, wherein said composition is a homeopathic gel or cream.

20. (canceled)

21. The method of claim 1, further comprising administering an additional treatment modality comprising a second SL-containing composition, an adjuvant, an inhibitor, tape-stripping, cryotherapy, electrosurgery, curettage, laser therapy, salicylic acid, trichloroacetic acid, podophyllin, cantharidin, 5-fluorouracil, bleomycin, topical imiquimod, intralesional Candida antigen, topical squaric acid dibutyl ester, oral cimetidine, surgical lesion removal, electrodesiccation, lasers of various wavelengths (ablative and non-ablative), radio-frequency ablation, dermabrasion, curettage, surgical excision, an ablative, a chemical peeling agent, disturbing the surface of the lesion, decreasing the thickness or size or overall volume of the lesion, increasing the surface area of the lesion, or a combination thereof.

22. The method of claim 6, wherein the wart or virus-associated skin or mucosal lesion has previously received treatment.

23. The method of claim 6, wherein the wart or virus-associated skin or mucosal lesion has not previously received treatment.

24. The method of claim 6, wherein the wart is a recurrent wart or a wart-associated skin or mucosal lesion.

25. The method of claim 6, wherein the wart or wart-associated skin or mucosal lesion has persisted in situ for 6 months, 12 months, 2 years, 3 years, 4 years, 5 years or longer.

26. The method of claim 6, wherein the wart or wart-associated skin or mucosal lesion is reduced in depth by 50%, 75%, or 90% or more after three weeks of daily treatment.

27-28. (canceled)

29. The method of claim 6, wherein the wart or wart-associated skin or mucosal lesion is reduced in depth by 50%, 75% or 90% or more after six weeks of daily treatment.

30-31. (canceled)

32. The method of claim 6, wherein the wart or wart-associated skin or mucosal lesion is reduced in volume by 50%, 75%, or 90% or more after three weeks of daily treatment.

33-34. (canceled)

35. The method of claim 6, wherein the wart or wart-associated skin or mucosal lesion is reduced in volume by 50%, 75% or 90% or more after six weeks of daily treatment.

36-37. (canceled)

38. The method of claim 6, wherein the wart or wart-associated skin or mucosal lesion remains reduced in diameter or volume by at least 50%, 75% or 90% for at least three months following treatment.

39-40. (canceled)

41. A kit for treating a wart or wart-associated skin or mucosal lesion comprising a container comprising greater than 0.0001% w/w to about 10% w/w Helenalin (H. tox; (±)-4-Hydroxy-4a,8-dimethyl-3,3a,4a,7a,8,9,9a-octahydroazuleno[6,5-b]furan-2,5-dione or its derivatives).

42-45. (canceled)

46. A dermal or mucosal covering impregnated with Helenalin (H. tox; (±)-4-Hydroxy-4a,8-dimethyl-3,3a,4a,7a,8,9,9a-octahydroazuleno[6,5-b]furan-2,5-dione or its derivatives).

47-50. (canceled)
